(19) United States
(12) Patent Application Publication
(10) Pub. No.: US 2022/0189589 A1
(43) Pub. Date: Jun. 16, 2022

(54) HIGHLY RELIABLE DATA TRANSACTION SYSTEM, AND HIGHLY RELIABLE DATA TRANSACTION METHOD

(71) Applicant: HUMAN'S DATA LIMITED, Tokyo (JP)

(72) Inventors: Akito OGAWA, Tokyo (JP); Yoshihide NAGASE, Tokyo (JP); Takeshi KUBODERA, Tokyo (JP)

(21) Appl. No.: 17/441,881

(22) PCT Filed: Mar. 30, 2020
(86) PCT No.: PCT/JP2020/014684

§ 371 Date: Sep. 22, 2021

(30) Foreign Application Priority Data
Mar. 29, 2019 (JP) 2019-069205

(51) Int. Cl. G16H 10/40; G16H 10/60; G16H 50/30; G06Q 40/08

(52) U.S. Cl. CPC G16H 10/40; G06Q 40/08; G16H 50/30; G16H 10/60

(57) ABSTRACT

PROBLEM TO BE SOLVED: To provide a highly reliable data transaction systems with a distributed network, and a highly reliable data transaction method.

MEANS TO SOLVE THE PROBLEM: A highly reliable data transaction system with a distributed network has an authentication means for authenticating a health check institution, a first acquisition means for acquiring health check information of an examinee, a highly reliable data generation means for generating highly reliable data related to the examinee, a first recording means for recording the generated highly reliable data in blocks connected by a blockchain built in the distributed network, and a first delivery means for delivering part or all of the highly reliable data to the user, depending on the transaction information.

### TECHNICAL FIELD

The present invention relates to a highly reliable data transaction system, which has a distributed network, and which transacts the health check results of examinees, and a highly reliable data transaction method.

### BACKGROUND ART

Heretofore, as technologies for transmitting address data on a blockchain to the provider's side, to allow the provider to reference access data from the source, and acquire and utilize the personal medical data stored in the storage at the source have been proposed, including, for example, the method disclosed in patent literature 1, which is a method to use blockchains to acquire and utilize data that relates to personal information, by means of an information processing device used by individuals.

Patent literature 1 discloses a medical information utilization system for utilizing medical data, in which a plurality of types of transaction data (TR), each being different medical data, are acquired from different institutions, and stored in the user's secret distributed storages, so that the data is consolidated and new master data (MA) is generated, on an as-needed basis.

There are a plurality of different types of medical data related to the mind and body of individuals. For example, there are medical data that is stored in the form of in-hospital databases in health check institutions such as hospitals, medical data that is stored in medical data clouds accessed and used by health check institutions, and medical data that is stored by business companies that provide personal healthcare, and consent for exchanging electronic data with cloud systems and database systems that serve as information resources of these medical data is obtained in advance, so that individuals can gather and manage their personal medical data scattered in various places/services.

### CITATION LIST

### Patent Literature

Patent Literature 1: Japanese Unexamined Patent Application Publication No. 2018-109994

### SUMMARY OF INVENTION

### Problem to be Solved by the Invention

Now, for example, in the method according to patent literature 1, a plurality of various types of data are targeted and made available for use, but when, for example, trying to use medical data from an individual's health check, to use new services or to develop completely new products, a variety of data might exist inter-connected on a blockchain. That is, although the data's history is known, it is difficult to make sure that the medical data that has been examined in a proper health check institution, or that the examination result has not been tampered with, at the time of the first recording, and credibility is required when newly recorded on the blockchain.

Furthermore, when trying to use medical data for those services or for developing completely new products, the medical data to serve as a basis then has different data formats and includes no correction data such as reference values and standard values that can be compared, and therefore is an obstacle when analyzing a plurality of medical data together and getting effective results. Consequently, the problem is that medical data that is prepared by taking into account the performance and functional characteristics of medical devices used for medical examinations at other health check institutions is difficult to process, and therefore results in lower workability.

Furthermore, given the above, it is not possible to prepare for new services, product development, transactions and so forth that are suitable for users, and therefore not possible to provide or promote high-value-added products and services, financial services and so forth that are suitable for users. Furthermore, no framework has been constructed, in which the user himself/herself can enjoy a wide range of benefits by providing his/her own medical data, at his/her own will, to users who need it.

So, the present invention has been made in view of the above-mentioned problems, and it is therefore an object of the present invention to provide a highly reliable data transaction system and a highly reliable data transaction method that can ensure the reliability of data recorded on a blockchain, based on the will of examinees, shorten the working time required for analyzing and processing the data to use, and facilitate the provision and development of services and products that are suitable for both examinee and users, high value-added products and services, financial services, and so forth.

### Means for Solving the Problems

The highly reliable data transaction system according to the first invention is a highly reliable data transaction system with a distributed network, transacting a health check result of an examinee, the highly reliable data transaction system having an authentication means for authenticating a health check institution, based on health check institution information related to the health check institution where the examinee takes a health check, a first acquisition means for acquiring, at least, examinee information related to the examinee taking the health check at the health check institution authenticated by the authentication means, consent information related to consent for transaction of a health check condition for the health check and a health check result of the health check, and health check result data of the examinee having taken the health check at the health check institution, a highly reliable data generation means for generating highly reliable data, subject to the transaction with a user, from the health check result data of the examinee acquired in the first acquisition means, based on authentication information indicating that the health check institution is authenticated, and the consent information, a first recording means for sorting and recording part or all of the highly reliable data, generated in the highly reliable data generation means, in a highly reliable data storage database for storing the highly reliable data, or sorting and storing the highly reliable data, generated in the highly reliable data generation means, in blocks connected by a blockchain built in the distributed network, and a first delivery means for, when transaction information related to acquisition of the part or all of the highly reliable data recorded by the first recording means is acquired, delivering the part or all of the highly reliable data to the user, depending on the transaction information.

Based on the first invention, the highly reliable data transaction system according to a second invention further includes a second acquisition means for acquiring use condition information related to a condition of use of the highly reliable data, via an examinee terminal of the examinee, a receiving means for receiving use request information related to a use request for the highly reliable data, from a user terminal of the user requesting use of the highly reliable data, an access right granting means for granting an access right, to the user, to the part or all of the highly reliable data recorded by the first recording means, via the examinee terminal, based on the use condition information acquired in the second acquisition means and the use request information received in the receiving means, and a second delivery means for transacting the part or all of the highly reliable data, where the access right is granted by the access right granting means, based on the use condition information, the use request information and the access right, and, furthermore, delivering the part or all of the highly reliable data, to the user, depending on a result of the transaction.

Based on the first invention or the second invention, in the highly reliable data transaction system according to a third invention, the highly reliable data generation means further includes a standardization process means for standardizing part or all of the health check result data acquired in the first acquisition means, a second recording means for recording the health check result data, standardized in the standardization process means, in the highly reliable data storage database or in the blocks connected by the blockchain built in the distributed network, as standardized health check result data, a third delivery means for, when the transaction information related to the acquisition of the part or all of the highly reliable data recorded by the first recording means is acquired from the user, delivering the part or all of the highly reliable data to the user, depending on the transaction information.

Based on the first invention, the highly reliable data transaction system according to a fourth invention further includes a financial data recording unit for recording financial data, related to billing of a health check fee for the health check institution, in the blocks connected by the blockchain built in the distributed network related to financing of the highly reliable data, a claim information delivery means for gathering medical care claims of a same debtor, among medical care claim information of a same health check institution, based on the health check institution information, health check fee debtor information, an amount of claims, and a due date of payment, included in the financial data recorded by the financial data recording unit, and delivering these to a financial institution as one or more credit collateral information, and a transaction determining means for allowing the health check institution or an agent of the health check institution to select a financial institution where funding through the health check institution is possible, and to determine assignment of claims by the selected financial institution, discount of claims, or the staff in charge of claims, depending on the result of the delivery of the credit collateral information from the claim information delivery means.

Based on the first invention or the fourth invention, in the highly reliable data trading system according to a fifth invention, the transaction determining means collects the medical care claims of the same debtor, among the medical care claim information of the same health check institution, based on the health check institution information, the health check fee debtor information, the amounts of claims, and the due dates of payment, gathers the credit collateral information, and executes cloud lending based on one or more credit collateral information.

Based on the first invention or the fourth invention, in the highly reliable data transaction system according to a sixth invention, the second acquisition means acquires estimation information related to medical care of the examinee, together with the authentication information, from a medical care health check institution, records content subject to support for the treatment of the examinee, in the blocks connected by the blockchain built in the distributed network, via the examinee terminal, based on the estimation information acquired in the second acquisition means and the highly reliable data of the examinee recorded by the first recording means, and executes a transaction with a cloud founder, a cloud lender or the financial institution, based on the estimation information related to the medical care of the examinee, depending on the recorded content subject to support.

Based on the first invention according to any one of the first invention to the third invention, the highly reliable data transaction system according to a seventh invention further includes a right acquisition means for acquiring a right to allow the examinee to receive an intangible or tangible benefit, depending on the delivery of the part or all of the highly reliable data by the first delivery means, the second delivery means, or the third delivery means, and an collecting means for collecting compensation in accordance with the right acquired in the right acquisition means, from the health check institution or the user.

Based on one of the first invention to the third invention, the highly reliable data transaction system according to an eighth invention further includes a third recording means for acquiring track information and history information related to a result of the delivery, based on the delivery of the part or all of the highly reliable data to the user, and recording the acquired track information and history information in the blocks connected by the blockchain built in the distributed network.

Based on the first invention, in the highly reliable data transaction system according to a ninth invention, the examinee information acquired in the first acquisition means includes, at least, health check data related to the health check of the examinee, medical care data related to the medical care of the examinee, medication data related to the medication of the examinee, genetic data related to the examinee, medical measurement device information related to the medical measurement device for performing the health check, fee billing data related to the fee, and insurer data related to the insurer of the examinee.

Based on the first invention, in the highly reliable data transaction system according to a tenth invention, the first acquisition means acquires, from the health check institution, the examinee information of the examinee and correction information related to correction of the health check result data of the examinee, the highly reliable data generation means generates corrected highly reliable data of the highly reliable data generated, based on the correction information acquired in the first acquisition means, and the first recording means adds and records part or all of the corrected highly reliable data, generated in the highly reliable data generation means, to the highly reliable data storage database or each block connected by the blockchain built in the distributed network.

Based on the first invention, in the highly reliable data transaction system according to an eleventh invention, the health check institution authenticated by the authentication means includes, at least, a health check institution that provides health check data, a medical care institution that provides medical care data, a medical care institution that provides various medical data, a testing institution that provides test information, a dental institution that provides dental data, a medicine institution that provides medicine data, or a gene institution that provides genetic data.

Based on the first invention, in the highly reliable data transaction system according to a twelfth invention, the highly reliable data generated in the highly reliable data generation means includes, at least, authenticator information related to an authenticator performing the authentication through the authentication means, authentication information including date/time information to show the date and time of the authentication, consent information of the examinee, health check institution information for identifying the health check institution where the examinee takes the health check, medical device information for acquiring the health check result data, assignment information related to correction of standardization, use information related to use of the highly reliable data, attribute-specific reference value comparative standard deviation data provided per examinee attribute, aging transition data of the examinee, storage location information for identifying a location where the health check result data is stored separately, my-number information for identifying the examinee, health insurance card information of the examinee, or future predicting data of the examinee.

Based on the first invention, in the highly reliable data transaction system according to a thirteenth invention, the assignment information included in the highly reliable data generated in the highly reliable data generation means includes, at least, standard deviation correction data for the health check result data, attribute-specific reference value comparative standard deviation data, transition data, analysis data, device information or common language that serves as a standard for standardization, and standardization model data.

Based on the first invention, the highly reliable data transaction system according to the fourteenth invention further includes a third acquisition means for acquiring the highly reliable data generated in the highly reliable data generation means, a reference database, in which a plurality of associations corresponding to weight variables between a plurality of data included in past highly reliable data acquired in advance, and a plurality of data included in reference information linked with the past highly reliable data, is stored, an evaluation means for consulting the reference database and generating an evaluation result of the highly reliable data, a notification means for extracting information to provide and a destination where the information is provided, based on the evaluation result in the evaluation means, and, furthermore, notifying the extracted destination of the evaluation result and the information to provide, and an updating means for acquiring the highly reliable data and the corrected highly reliable data recorded in the blocks connected by the blockchain by the third recording means, based on the result of the notification by the notification means, updating corresponding history information, and storing as a new block.

Based on the fourteenth invention, in the highly reliable data transaction system according to a fifteenth invention, the training model is built on machine learning, using the past highly reliable data and the reference information as training data.

The highly reliable data transaction method according to a sixteenth invention is a highly reliable data transaction method with a distributed network, transacting a health check result of an examinee, the highly reliable data transaction method including an authentication step of authenticating a health check institution, based on health check institution information related to the health check institution where the examinee takes a health check, a first acquisition step of acquiring, at least, examinee information related to the examinee taking the health check at the health check institution authenticated in the authentication step, consent information related to consent for transaction of a health check condition for the health check and a health check result of the health check, and health check result data of the examinee having taken the health check at the health check institution, a highly reliable data generation step of generating highly reliable data, subject to the transaction with a user, from the health check result data of the examinee acquired in the first acquisition step, based on authentication information indicating that the health check institution is authenticated, and the consent information, a first recording step of sorting and recording part or all of the highly reliable data, generated in the highly reliable data generation means, in a highly reliable data storage database for storing the highly reliable data, or sorting and storing the highly reliable data, generated in the highly reliable data generation step, in blocks connected by a blockchain built in the distributed network, and a first delivery step of, when transaction information related to acquisition of the part or all of the highly reliable data recorded in the first recording step is acquired, delivering the part or all of the highly reliable data to the user, depending on the transaction information.

### Advantageous Effects of Invention

According to the first invention to the fifteenth invention, the highly reliable data transaction system has a distributed network, and acquires authenticated health check information of an examinee. Consequently, it is possible to generate highly reliable data from the authenticated health check information, and store the generated highly reliable data in a blockchain, so that the highly reliable data can be used at the will of the examinee. By this means, the reliability of highly reliable data acquired in authenticated health check institutions can be ensured, and it is possible to facilitate the development of services and products suitable for both examinees and users, and the provision of products and services, as well as financial services, with high added transaction values.

In particular, according to the first invention, the highly reliable data generation means generates highly reliable data of the examinee based on authentication information and health check information. Consequently, highly reliable data can be generated based on authentication information of an authenticated medical care institution and the medical care result of the examinee. By this means, the credibility of medical care results and authentication information can be ensured. Furthermore, it is possible to facilitate the development of services and products suitable for both examinees and users, and the provision of products and services, as well as financial services, with high added transaction values.

In particular, according to the first invention, the first recording means records part or all of the highly reliable data generated in the highly reliable data generation means in blocks connected by blockchains built in the distributed network. Consequently, health check results from various institutions authenticated by authentication information assignment institutions, and part or all of the highly reliable data, are recorded on the blockchains. By this means, part or all of the highly reliable data can be used, and the working time required for the analysis and processing can be shortened. Furthermore, the developers of new services, completely different product development and so forth can feel safe to use highly reliable data.

Also, according to the second invention, the second acquisition means acquires the use condition information for highly reliable data, satisfying the examinee's highly reliable data, from the examinee terminal, at the will of the examinee. Consequently, it is possible to present the conditions of use to suit the examinee's request, to the user terminal requesting the use of highly reliable data. By this means, the user terminal can confirm the use condition information of each highly reliable data and acquire the desired highly reliable data efficiently, before analyzing the highly reliable data on a collective scale. Furthermore, it is possible to facilitate the development of services and products suitable for both examinees and users, and the provision of products and services, as well as financial services, with high added transaction values.

In particular, according to the second invention, the second recording means records part or all of the highly reliable data generated in the highly reliable data generation means in blocks connected by a blockchain built in the distributed network, and, furthermore, records all records, including corrections, updates, deletions and so forth, only when required by the authentication information assignment institution. Consequently, the highly reliable data, which includes the health check information authenticated by the authentication information assignment institution, is first recorded on the blockchains, and then all the subsequent corrections and updates are recorded and delivered. By this means, the developers of new services, completely different product development and so forth can feel safe to use part or all of the highly reliable data. Furthermore, it is possible to facilitate the development of services and products suitable for both examinees and users, and the provision of products and services, as well as financial services, with high added transaction values.

Also, according to the third invention, the standardization process means standardizes part or all of the health check result data based on the conditions and consent included in the transaction information. Consequently, it is possible to transact standardized highly reliable data. By this means, it is possible to ensure the comparability and reliability of transacted data, and develop services and products that are suitable for customers. Furthermore, it becomes possible to execute transactions based on the use condition information, so that it is possible to facilitate the development of services and products suitable for both examinees and users, and the provision of products and services, as well as financial services, with high added transaction values.

Also, according to the fourth invention, the financial data recording unit records the standardized health check result data, and, at the same time, records the financial data related to the billing of the health check fee for the health check institution, in blocks connected by a blockchain built in the distributed network for the financing of highly reliable data. Consequently, it is possible to collect the medical care claims of the same debtor among the medical care claim information of the same health check institution, based on the health check institution information, the health check fee debtor information, the amounts of claims, and the due dates of payment, and deliver the collected credit collateral information to the financial institution as credit collateral information that is based on financial data that is certain to exist. By this means, it is possible to provide high-quality financial services to medical care institutions, thereby encouraging the use of the system, and, as a result of this, it is possible to promote services and product development suitable for both examinees and users, and products and services with high added value in transactions.

Also, according to the fifth invention, the transaction determining means collects the medical care claims of the same debtor, among the medical care claim information of the same health check institution, based on health check institution information, debtor information of the insurer, the amounts of claims, and the due dates of payment, makes the collected credit collateral information into a fund, and executes cloud lending that can secure credit collateral, based on the funded credit collateral information. Consequently, it is possible to carry out transactions by cloud lending that is part from financial institutions. By this means, it is possible to provide high-quality financial services to medical care institutions, thereby encouraging the use of the system, and, as a result of this, it is possible to promote services and product development suitable for both examinees and users, and products and services with high added value in transactions. Also, it is possible to receive various financial services, not only from financial institutions, but also from a wide range of people.

Also, according to the sixth invention, it is possible to acquire estimation information related to the medical care of the examinee, together with the authentication information, from the medical care institution, and the examinee can specify the target case of cloud funding, via the examinee terminal, based on the estimation information acquired in the second acquisition means and the highly reliable data of the examinee recorded by the first recording means, and executes transactions with the cloud founder, cloud lender or financial institutions, based on the estimation information related to the medical care of the examinee, depending on the content that is subject to support. By this means, it is possible to collect donations for high medical care and treatment costs of the examinees, or provide high-quality financial services, thereby encouraging the use of the system, and, as a result of this, it is possible to promote services and product development suitable for both examinees and users, and products and services with high added value in transactions. Also, it is possible to receive various financial services, not only from financial institutions, but also from a wide range of people.

Also, according to the seventh invention, the right acquisition means acquires the right to allow the examinee and the transaction manager to receive intangible or tangible benefits, depending on the delivery of part or all of highly reliable data by the first delivery means, the second delivery means, or the third delivery means. Consequently, the compensation in accordance with the right can be collected from the health check institution or the user. By this means, the examinee is able to secure a mechanism whereby the examinee gets his/her own benefits, so that it is possible to facilitate the development of services and products suitable for both examinees and users, and the provision of products and services, as well as financial services, with high added transaction values.

Furthermore, according to the eighth invention, the third recording means acquires track information and history information related to the result of delivery, based on the delivery of part or all of highly reliable data to the user, and records the acquired track information and history information in blocks connected by a blockchain built in the distributed network. Consequently, highly reliable data can be used for analysis, processing and so forth. By this means, it is possible to make people other than the examinee or unrelated to the initial health check providing institution unable to tampered with the medical data, so that it is possible to facilitate the development of services and products suitable for both examinees and users, and the provision of products and services, as well as financial services, with high added transaction values.

Also, according to the ninth invention, the examinee information acquired in the first acquisition means includes, at least, health check data related to the health check of the examinee, medical care data related to the medical care of the examinee, medication data related to the medication of the examinee, genetic data related to the examinee, medical measurement device information related to the medical measurement device for performing the health check, fee billing data related to the fee, and insurer data related to the insurer of the examinee. Consequently, it is possible to use highly reliable data in new services using highly reliable data and in development of completely different products. By this means, the developers of new services or completely different product development can use highly reliable data for analysis and processing, even if the highly reliable data to be compared is in different formats.

Also, according to the tenth invention, the first acquisition means acquires examinee information of the examinee and correction information related to the correction of the health check result health check data of the examinee, from the health check institution. Consequently, it is possible to generate corrected highly reliable data of the generated highly reliable data, based on the acquired correction information. By this means, the developers of new services or completely different product development can use highly reliable data for analysis and processing, even if the highly reliable data to be compared is in different formats.

Also, according to the eleventh invention, the authentication means authenticates the health check institution. Consequently, at least a medical care institution that provides health check data, a medical care institution that provides medical care data, a medical care institution that provides various medical data, a testing institution that provides test information, a dental institution that provides dental data, a medicine institution that provides medicine data, or a gene institution that provides genetic data can be authenticated. By this means, the workability is improved. Furthermore, it is possible to facilitate the development of services and products suitable for both examinees and users, and the provision of products and services, as well as financial services, with high added transaction values.

Also, according to the twelfth invention, the highly reliable data generation means generates highly reliable data. Consequently, it is possible to include, at least, authenticator information related to the authenticator performing the authentication through the authentication means, authentication information including date/time information to show the date and time of the authentication, consent information of the examinee, health check institution information for identifying the health check institution where the examinee takes the health check, medical device information for acquiring the health check result data, assignment information related to the correction of standardization, use information related to the use of the highly reliable data, attribute-specific reference value comparative standard deviation data provided per examinee attribute, aging transition data of the examinee, storage location information for identifying the location where the health check result data is stored separately, my-number information for identifying the examinee, health insurance card information of the examinee, or future predicting data of the examinee. By this means, the workability is improved. Furthermore, it is possible to facilitate the development of services and products suitable for both examinees and users, and the provision of products and services, as well as financial services, with high added transaction values. Furthermore, highly reliable data over the life of the examinee is recorded, so that, a wide range of financial support can be received by complaints from the examinee or his/her family, regarding the burden of large medical expenses such as advanced medical care. Since the data is highly reliable, the fund provider can also feel safe to provide funds.

Also, according to the thirteenth invention, the highly reliable data includes assignment information. Consequently, it is possible to include, at least, standard deviation correction data for the health check result data, attribute-specific reference value comparative standard deviation data, transition data, analysis data, device information or common language that serves as a standard for standardization, and standardization model data. By this means, the workability is improved. Furthermore, it is possible to facilitate the development of services and products suitable for both examinees and users, and the provision of products and services, as well as financial services, with high added transaction values.

Also, according to the fourteenth invention, the evaluation means generates an evaluation result of highly reliable data. Consequently, the evaluation result and information that is provided based on the evaluation result can be notified to the provider. By this means, it is possible to acquire highly reliable data and corrected highly reliable data based on the result of notification, and update history information, so that it is possible to ensure the reliability of the highly reliable data. Furthermore, it is possible to facilitate the development of services and products suitable for both examinees and users, and the provision of products and services, as well as financial services, with high added transaction values.

Also, according to the fifteenth invention, the training model is built on machine learning using past highly reliable data and reference information as training data. Consequently, in addition to the huge amount of highly reliable data in the past, it is possible to render quantitative evaluation even when evaluating unknown highly reliable data different from them. By this means, it is possible to further improve the accuracy of evaluation. Furthermore, it is possible to facilitate the development of services and products suitable for both examinees and users, and the provision of products and services, as well as financial services, with high added transaction values.

Also, according to the sixteenth invention, the highly reliable data transaction method generates highly reliable data of the examinee based on authentication information and health check information. Consequently, it is possible to generate highly reliable data based on authentication information of authenticated medical care institutions and the medical care result of the examinee. By this means, it is possible to ensure the credibility of medical care results and authentication information. Furthermore, it is possible to facilitate the development of services and products suitable for both examinees and users, and the provision of products and services, as well as financial services, with high added transaction values.

### BRIEF DESCRIPTION OF DRAWINGS

FIG. 1 is a schematic diagram to show an example of the configuration of a highly reliable data transaction system according to one embodiment;

FIG. 2 is a schematic diagram to show an example of the configuration of an examinee terminal according to one embodiment;

FIG. 3 is a schematic diagram to show an example of acquisition of authentication information according to one embodiment;

FIG. 4 is a schematic diagram to show an example of granting of access rights according to one embodiment;

FIG. 5A is a schematic diagram to show examples of functions of a highly reliable data transaction system according to one embodiment, and FIG. 5B is a schematic diagram to show examples of functions of a highly reliable data transaction system according to one embodiment;

FIG. 6 is a schematic diagram to show an example of a transaction management information database of a transaction management system according to one embodiment;

FIG. 7 is a schematic diagram to show an example of a medical management database of a health check institution system according to one embodiment;

FIG. 8 is a schematic diagram to show an example of an authentication information assignment database of an authentication information assignment system according to one embodiment;

FIG. 9 is a schematic diagram to show an example of a highly reliable data storage database of a highly reliable data storage system according to one embodiment;

FIG. 10 is a schematic diagram to show an example of hashing of acquired data according to one embodiment of the present invention;

FIG. 11 is a schematic diagram to show an example of data management in a blockchain in a management server according to one embodiment of the present invention;

FIG. 12 is a schematic diagram to show an example of the operation for adding hash data in a data management device according to one embodiment;

FIG. 13 is a schematic diagram to show an example of a reference database according to one embodiment;

FIG. 14 is a flowchart to show an example of the operation for acquiring authentication information in a highly reliable data transaction system according to one embodiment;

FIG. 15 is a flowchart to show an example of the operation for granting access rights in a highly reliable data transaction system according to one embodiment; and

FIG. 16 is a schematic diagram to show an example of delivery of standardized health check result data according to one embodiment.

### DESCRIPTION OF EMBODIMENTS

Hereinafter, an example of a highly reliable data transaction system according to one embodiment of the present invention will be described with reference to the drawings.

### Embodiment: Configuration of Highly Reliable Data Transaction System 1

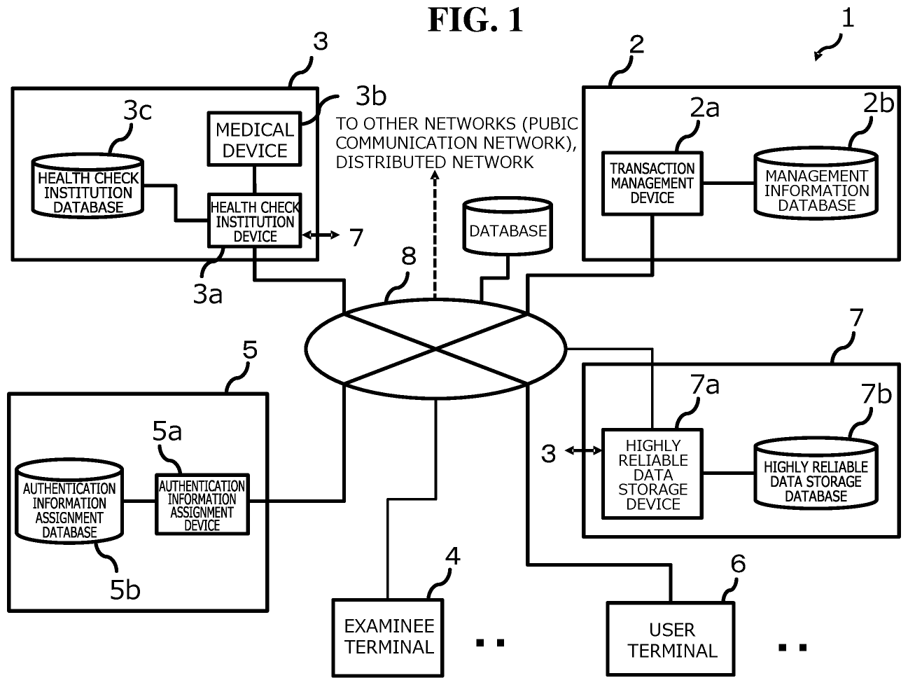

An example of the configuration of a highly reliable data transaction system 1 according to one embodiment will be described below with reference to FIG. 1. FIG. 1 is a block diagram to show an overall configuration of a highly reliable data transaction system 1 according to one embodiment.

As shown in FIG. 1, the highly reliable data transaction system 1 is comprised of, for example, a transaction management system 2, a plurality of health check institution systems 3, a plurality of examinee terminals 4, a plurality of authentication information assignment systems 5 for authenticating each health check institution, a plurality of user terminals 6, and a highly reliable data storage system 7. The transaction management system 2 is connected, via a distributed network 8 (blockchain network), with a plurality of health check institution systems 3, taking part in the distributed network 8, a plurality of examinee terminals 4, a plurality of authentication information assignment systems 5, a plurality of user terminals 6, a highly reliable data storage system 7 and so forth, and highly reliable data transaction services take place.

Here, “highly reliable data” (or highly reliable medical data) refers to data that is provided by authenticated or trusted health check institutions. The types of highly reliable data include, for example, highly reliable data (medical examination/medical care data, etc.) acquired in institutions that provide medical data, such as health check institutions (medical care institutions such as hospitals), testing institutions or the like, medical examination/examinee consent data, highly reliable data acquired in institutions that provide medical data, such as health check institutions (for example, medical care institutions, hospitals, etc.) and testing institutions, highly reliable data of examinees that is acquired, highly reliable data of users that is transacted, correction data of various highly reliable data, and so forth, each stored in a blockchain and various databases.

The distributed network 8, using known blockchain technologies, for example, keeps the records (blocks) of transactions that occur between each system connected via the distributed network 8, as chained blocks. Each transaction's block stores, inside that block, the hash value of the content of the previously-generated block, in addition to the content of that transaction, and is recorded as a legitimate block connected in a chain.

Also, in the distributed network 8, part or all of highly reliable data may be directly recorded on blockchains, and, besides, all the data, information and so forth of the server of the corresponding institution where health check results, health check data, highly reliable data and so forth are recorded, and the tracks of their records, may be recorded on the blockchains.

The types of blockchains include, besides the highly reliable data blockchain for recording highly reliable data, for example, a highly reliable data blockchain for recording information related to the examinees, whose highly reliable data is subject to transaction, and the health check data of the examinees. The highly reliable data transaction system 1 may further include a highly reliable medical financial data blockchain, which, based on the highly reliable data blockchain, records part or all of the highly reliable data to be transacted, or information related to various institutions of highly reliable data, transaction information, and the like.

The reliability of the highly reliable data blockchain is ensured by the fact that it is based on a highly reliable data blockchain that stores highly reliable data. Furthermore, the security and reliability of the highly reliable medical financial data are ensured by the fact that it is based on a highly reliable data blockchain that stores highly reliable data.

The highly reliable blockchain and the highly reliable medical data blockchain, and the highly reliable medical data blockchain and the highly reliable medical financial data blockchain are linked with each other by highly reliable data, examinees, and the like. The types of data stored in these blockchains will be described later.

Individual transactions are carried out using P2P (Peer to Peer) communication, but, in one embodiment, communication other than P2P communication may be used. The data to be transacted is, for example, highly reliable data of an examinee himself/herself, in one embodiment, but other data may include different types of data as long as these are information or data that relate to individuals, such as nursing data, lifestyle data and so forth. Also, in addition to an examinee himself/herself, for example, family information such as information about the examinee's family may be used. Transaction data is provided based on blockchain technology as mentioned earlier, and, by using known hash values and public key cryptosystem, the security of transaction data is ensured.

<Transaction Management System 2>

The transaction management system 2 according to one embodiment includes, for example, a transaction management device 2a that manages a highly reliable data transaction management system using blockchains, and a transaction management information database 2b that records various information related to transactions. The transaction management device 2a is used, for example, by the transaction manager of the transaction management system 2. Note that the configuration of each table of the transaction management information database 2b provided in the transaction management system 2 will be described later.

The transaction management system 2 holds various information related to each participant (health check institution, medical care institution, medication institution, authentication information assignment institution, examinees, users, etc.) of transaction services of highly reliable data, and carries out transactions of highly reliable data m between each participant, via the distributed network 8. The transaction management system 2 makes a transaction with a prospective user wishing to use an examinee's highly reliable data, based on the access rights to the examinee's highly reliable data, set by the examinee via the examinee terminal 4.

The highly reliable data to be transacted is recorded in blocks connected by the blockchains built in the distributed network 8, and may be stored in the highly reliable data storage database 7b of the highly reliable data storage system 7. As for the highly reliable data to be transacted, only the highly reliable data that relates to the examinee, or part or all of the highly reliable data requested for use, may be provided to the user, via the highly reliable data storage device 7a, depending on the access rights set by the examinee.

In the transaction management system 2, highly reliable data is transacted between the examinee and the user via the blockchains built in the distributed network 8, and the history of a series of transactions is recorded in blocks connected by blockchains. That is, information such as transactions recorded in the blocks connected by the blockchains built in the distributed network 8 is recorded in each institution's database or the like, in a distributed manner.

The blockchains may include, besides the blockchain for recording the highly reliable data that is transacted, a highly reliable medical data blockchain (not shown) for storing various medical data, including, for example, the examinee's health check institution, the personnel at the health check institution, the medical care fee, receipt information, and so forth.

Also, the blockchains may include a highly reliable medical financial data blockchain (not shown) that is based on the highly reliable data blockchain or a highly reliable medical data blockchain. In the highly reliable medical financial data blockchain, based on the highly reliable data blockchain or the highly reliable medical data blockchain, various finance-related data and information are recorded in blocks connected to the blockchains. Recording in each blockchain may be performed via the highly reliable data storage system 7, or may be performed by each health check institution.

In the transaction management system 2, for example, the conditions of use and the like to apply to the transactions of highly reliable data for each examinee, desired conditions and the like regarding the transactions of highly reliable data for each user, and so forth are stored in the transaction management information database 2b of the transaction management system 2, which will be described later, via service sites on the Web that enable transactions of highly reliable data of examinees for each examinee (homepages for exclusive use of individual examinees, my-page, etc.).

The transaction management system 2 consults the transaction management information database 2b, and, based on various information referenced, carries out secure transactions via the blockchains built in the distributed network 8.

The transaction management system 2 receives as input, for example, additional information (for example, request for the desired place of examination, the date and time, the items to test, etc.) regarding the transactions of highly reliable data, from an examinee's my-page, via the examinee's examinee terminal 4, so that additional information such as information about recommended health check institutions, additional examination services (for example, complete medical checkup) and the like can be registered. The examinee can consult this my-page via the examinee terminal 4 and check the registration information, appointment information, and so forth, on the screen.

As for the conditions of use for the transactions of highly reliable data for each examinee, for example, health check medical data, certified by a health check or medical care institution and stored in a blockchain, its receipt data (medical care fee bill/medical care fee invoice data), and the method of providing it may be specified. The transaction management system 2 provides the highly reliable data to be transacted, via the examinee terminal 4, based on the examinee's will (for example, agreement for taking a health check, the result of the health check, agreement for using and providing highly reliable data, etc.).

The transaction management system 2 enables the user to carry out transactions (cloud funding) for the cost of advanced medical care of the examinee, based on, for example, the health check result of the examinee's childhood cancer, heart disease, leukemia and so forth, transaction information, and/or the like. When doing so, the transaction management system 2 may collect the system use fee from the user. Furthermore, the transaction management system 2, for example, has advertisements from supporting companies, on each terminal (for example, examinee terminal, user terminal, etc.), and collects advertisement fees.

In the transaction management system 2, in the health check of the examinee, for example, the time frame, facility information, examination menu, and use fees that are available for medical care at each health check institution is acquired in advance, from a plurality of health check institutions (medical care institution) that provide health check services such as complete medical checkup. The transaction management system 2 may make a reservation for (or purchase) a free time period that is left, or a time period from, for example, a health check institution or the like.

The transaction management system 2 registers the time frame and reservation frame information for each health check institution acquired in this way, with the management information database 2b, in association with information for identifying each health check institution. The examinee consults these various registered information, and selects the health check institution of his/her choice.

The transaction management system 2 searches for the time frame at each health check institution (medical care institution) that matches the acquired additional information (request information) such as the desired location, date and time, and testing items, in response to the request from the examinee terminal 4. If there is information related to the matching health check institutions and time frames as a result of the search, the transaction management system 2 transmits that information to the examinee terminal 4 as result information (medical care candidate information).

The examinee terminal 4 displays the result information transmitted from the transaction management system 2, and displays the candidates on the screen, by adding a frame for concluding a contract with the health check institutions where a health check can be taken. For example, when a contract is concluded between an examinee and a complete medical checkup which the examinee has decided to use, the health check institution (medical care institution) to conduct the complete medical checkup may, with reference to past highly reliable data (medical data) received from the examinee, extracts additional medical care (health check) menu, and transmit this to the examinee's terminal.

Depending on the situation of progress of contract between the examinee and the health check institution, the health check institution and the highly reliable data transaction institution, the highly reliable data transaction institution and the user (for example, health insurance association, private agent, private PHR (Personal Health Record) business, company, etc.) and so forth, the transaction management system 2 may determine the fee (charge) and the appropriate amount of payment for transactions of highly reliable data (for example, collect 20% of the contract amount as a system use fee).

By this means, the transaction management system 2 enables the examinee to provide his/her information and take a complete medical checkup that is more suitable to him/her. In addition, the health check institution fills the vacant frames, and makes known and proposes higher value-added medical services that are customized for each examinee, by using personal health check data, at a lower cost, and on a wider scale, thereby contributing to improvement of sales by, for example, having new customers.

The transaction management system 2 may, when transacting highly reliable data of the examinee or confirming the examinee's data, prevent spoofing of the examinee himself/herself. The transaction management system 2 acquires, for example, identification information of the examinee's my-number card via a card reader (not shown).

The transaction management system 2 may access a my-number card database, based on the my-number card identification information, using known personal authentication technology, and identify the examinee based on the result of that access. By this means, the transaction management system 2 can authenticate the examinee who operates transactions on the terminal, so that it is possible to prevent the examinee from spoofing.

For example, in the transaction management system 2, only the examinee's highly reliable data that is set by the examinee himself/herself via the examinee terminal 4 may be subject to transactions. Desired conditions regarding the transactions of highly reliable data and the like are stored in the transaction management information database 2b of the transaction management system 2, which will be described later, and, with reference to the transaction management information database 2b, based on various information referenced, secure transactions are carried out through the blockchains built in the distributed network 8. By this means, transactions of highly reliable data that the examinee does not desired are not carried out.

For example, if there is highly reliable data of the examinee that has been forged or tampered with, the transaction management system 2 judges that it is not highly reliable data of the examinee that has been set by the examinee himself/herself via the examinee terminal 4, and/or that the desired conditions regarding the transactions of reliable data are not met, and does not transact the highly reliable data.

The transaction management system 2 records transactions via the blockchains by connecting them to blocks connected by the blockchains, together with exchanges and data between each system. In the transaction management information database 2b of the transaction management system 2, for example, information related to the transactions in each institution, examinee terminal 4, and user terminal 6, each connected via the distributed network 8, is stored in a distributed manner.

<Health Check Institution System 3>

The health check institution system 3 according to one embodiment acquires health check data (or medical care data, diagnosis data, medication data, test data, etc.) and health check result data of examinees in, for example, in medical care institutions, medication institutions, testing institutions, complete medical checkup, health management centers in companies and the like, in addition to systems such as general hospitals or university hospitals. In the health check institution system 3, for example, in the case of general hospitals, university hospitals and so forth, the examinee is examined and diagnosed by means of the health check institutional device 3a, such as medical examination equipment and diagnostic equipment provided in those hospitals.

The health check institution system 3 includes, for example, a health check institution device 3a, including various systems, equipment, applications and forth, provided in each health check institution, and operated by doctors, medical staff and the like, medical device 3b for performing various medical examinations for the examinee, and, in addition, a health check institution database 3c for storing various information such as examinee information handled by the health check institutions, medical record information of the examinee and so forth. Note that the configurations of the health check institution device 3a, the medical device 3b, and the health check institution database 3c provided in the health check institution system 3 will be described later.

Also, the health check institution system 3 according to one embodiment may provide answers to questions about various medical examinations from the examinee through the examinee device 4 of the examinee, for example, and making an appointment for a health check, sending a notice of a health check, confirming a health check may be possible in a certain period of time before the examination, using SNS that is available to the examinee for use, or AI chat or something that is provided by the health check institution system 3 and/or other institutions.

For example, after the examinee takes an examination or makes a reservation for an examination, the examinee can answer a medical questionnaire by the day of the examination (or on the day of the examination), by using an application (not shown) that the examinee can use on the examinee terminal 4. The answer data that is submitted is stored in the memory of the examinee terminal 4 or on the server of the institution where the examination takes place or where the examination is mediated, for example, the health condition of the examinee at the time of answering, with, for example, environment or surrounding-related data such as the temperature, humidity, weather, location information, information related to the examinee's posture at the time of answering and so forth, may be stored, in addition to the medical questionnaire and answers, for each examinee.

Various data of the examinee stored in the server or elsewhere may be transmitted to, for example, the health check institution where the examinee has made an appointment, by the day of the examination, based on the examinee's appointment information. Conventionally, when the examinee is given a medical questionnaire, the examiner has to give answers on the day of the examination, within a limited time, give answers that the examinee thinks fit to corresponding questions, as far as the memory of the examinee can go. It then naturally follows that the examinee's answers may not be always accurate.

For example, the examinee may receive as input, as appropriate, the situation (physical condition, life style, behavior, etc.) for a certain period before the examination, based on an appropriate date and time, place, timing, medical questionnaire and so forth, and the answers appropriately received as input by the examinee may be analyzed by AI (not shown) provided in the medical care system 3.

The health check institution system 3 is provided upon examination based on, for example, the result analyzed by AI or the like, and based on the appointment information of the examinee, the examination menu, the result of medical questionnaire, and so forth. By this means, for example, the health check institution system 3 makes it possible to provide high-value-added analytical information related to the examination of the examinee, before the day of the examinee's examination.

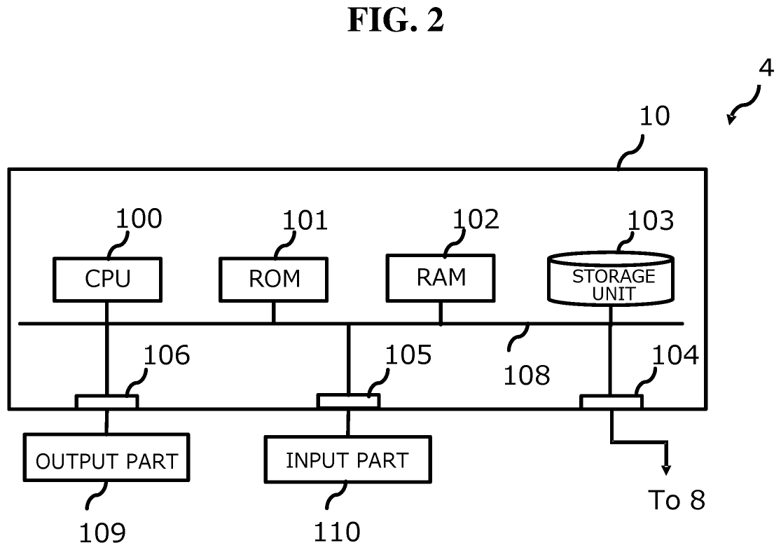

The health check institution device 3a may, for example, have the same configuration as the configuration of the examinee terminal 4 of FIG. 2, which will be described later, or the configuration of a client-server system, a cloud system, or the like (not shown), and include a housing (not shown), a CPU (Central Processing Unit), a ROM (Read Only Memory), a RAM (Random Access Memory), a storage unit, and a plurality of I/Fs, where each configuration is connected by an internal bus. The CPU controls the whole of the health check institution device 3a. The ROM stores operation codes for the CPU. The RAM is the task area for use when the CPU operates. The storage unit stores various information such as health check results, in addition to the examination-related information required for examinations. As for the storage unit, for example, a data storage device such as an SSD (Solid State Drive), a semi-conductor memory and the like may be used, in addition to an HDD (Hard Disk Drive).

The CPU is implemented by running programs stored in the storage unit and elsewhere, by using the RAM for the task area. Note that, for example, the health check institution device 3a may include a GPU (Graphics Processing Unit), which is not shown. Having a GPU enables faster calculation processing than usual.

The health check institution system 3 is introduced in, for example, a health check institution, a medical care institution and elsewhere, where examinations are performed by doctors, nurses, medical staff and so forth working at the health check institution, using various medical devices 3b via the health check institution device 3a, and various information such as medical records of examinees is stored in, for example, the health check institution database 3c.

The health check institution system 3 asks a predetermined authentication information assignment institution to authenticate a legitimate health check institution, and the authentication information assignment institution reviews this request, and authenticates the health check institution as being an authenticated institution. The health check institution system 3 assigns the authentication information, received from the authentication information assignment institution, to health check results. In addition to the authentication of its own health check institution, the health check institution system 3 may request authentication of the health check institution device 3a to use for medical examinations in the health check institution, authentication of the examinees, and so forth, to the predetermined authentication information.

For example, after the examinee takes an examination, the health check institution system 3 generates highly reliable data, including various information related to the examinee's examination (for example, personal identification information and consent information, authentication information that the health check institution is a certified health check institution, the health check result of the examinee and health check institution/medical device information set, correction information, etc.), and records the highly reliable data in the blocks connected by the blockchains built in the distributed network 8.

FIG. 2 is a schematic diagram to show an example of the configuration of the examinee terminal 4 according to one embodiment.

<Examinee Terminal 4>

The examinee terminal 4 sets the access rights. Using the examinee terminal 4, the examinee sets conditions such as, for example, the type and category of highly reliable data, what is to be acquired according to the examinee's will, and so forth.

Using the examinee terminal 4, for example, the examinee manages and utilizes his/her own highly reliable data (medical examination/medical care data, etc.), for his/her own benefit. The examinee sets the access rights to his/her health check result, health check data, or highly reliable data (medical examination/medical care data, etc.), via the examinee terminal 4. Based on the access rights set by the examinee terminal 4, the transaction management system 2 or the health check institution device 3a judges whether or not to carry out transactions, according to the conditions for transactions from the prospective users, and transacts and delivers the highly reliable data (medical examination/medical care data, etc.). Whether or not to carry out transactions of highly reliable data may be determined based on, for example, the first association stored in a reference database (not shown) built in advance using machine learning.

Using the examinee terminal 4, the examinee requests the health check institution device 3a to change and alter the examinee's setting information, conditions of use, and so forth, based on conditions such as, for example, the type and category of highly reliable data, what the examinee intends to acquire, and so forth. Note that the health check result, health check data, or highly reliable data of examinees who have already taken a health check at the health check institution device 3a cannot be changed by the examinee terminal 4. If there are such corrections or additions, they will be made by the health check institution device 3a, linked with various original information and data, and recorded in the blocks connected by or the blockchains built in the health check institution system 3 or the distributed network 8.

Based on the operation by the examinee, the examinee terminal 4, transacts the examinee's highly reliable data with the prospective users, through the transaction management system 2, in order to utilize the highly reliable data of the examinee, and manages his/her highly reliable data.

For the examinee terminal 4, for example, an electronic device such as a personal computer (PC) may be used, and, besides, an electronic device such as a smartphone or a tablet terminal, a chip with a communication function, a data rewriting function and a memory (for example, a microchip embedded in the body), a client-server type configuration using a LAN in the hospital or the like may be used. The examinee terminal 4 includes a housing 10, a CPU 100, a ROM 101, a RAM 102, a storage unit 103, and I/Fs 104 to 106. Each configuration 100 to 106 is connected by an internal bus 108.

The CPU (Central Processing Unit) 100 controls the whole of the examinee terminal 4. The ROM (Read Only Memory) stores operation codes for the CPU 100. The RAM (Random Access Memory) 102 is the task area for use when the CPU 100 operates. The storage unit 103 stores various information related to the medical examination the examinee has taken, information such as the URL of services provided by the transaction management system 2, personal information such as the examinee's profile, setting information, genetic data, and the like. As for the storage unit 103, for example, a data storage device such as an SSD (Solid State Drive), a hard disk, a semi-conductor memory and the like may be used, in addition to an HDD (Hard Disk Drive). Note that, for example, the health check institution device 3a may include a GPU (Graphics Processing Unit), which is not shown. Having a GPU enables faster calculation processing than usual.

The I/F 104 is an interface for transmitting and receiving various information on the blockchain connected via the distributed network 8. The I/F 104 transmits and receives various information to and from the transaction management system 2, the health check institution system 3, the authentication information assignment system 5, the user terminal 6, the highly reliable data storage system 7 and so forth, via the blockchains built in the distributed network. The I/F 105 is an interface for transmitting/receiving information to/from an input part 110. For example, a keyboard is used as the input part 110, and the user of the examinee terminal 4 inputs various information such as transmission/receipt or control commands for setting various information, via the input part 110. The I/F 106 is an interface for transmitting and receiving various information with an output part 109. The output part 109 outputs various information stored in the storage unit 103, the processing status of the examinee terminal 4, and the like. A display is used as the output part 109, and for example, a touch panel type may be used. Note that each of these functions may be controlled, for example, by artificial intelligence. Here, “artificial intelligence” may be based on any well-known artificial intelligence technology.

Also, the examinee terminal 4 communicates with, for example, a chip embedded in the human body, and acquires data stored in the chip (for example, genetic data, etc.). The chip is embedded in the body of the examinee in advance by, for example, a health check institution, and has an RFID function and the like.

The examinee terminal 4 may have a function such as RFID, and the data stored in the chip may be transmitted to another terminal or the like in response to an operation from the examinee terminal 4. The examinee terminal 4 may be capable of performing various processes for updating (adding, deleting, overwriting, etc.) the data stored in the chip, for example. Also, the data stored in the chip in the human body may be stored in the examinee information table of the health check institution database 3c of the health check institution 3a, for example.

The examinee information table stores, for example, personal information for identifying the person who takes the examination, consent information related to the consent between the person and the health check institution or transaction institution regarding the examination or transactions, the agreed date, the name of the health check institution or medical care/health check institution, the name of the insurer such as the health insurance association, various medical care/medical examination data handled by each institution or association, medical questionnaire data, receipt information, and so forth are stored. The examinee information table may be in a data format such as CSV format.

A plurality of various data, information and so forth related to the examinee are recorded in the examinee information table. In the examinee information table, for example, the results of transactions carried out by the transaction management system 2 are recorded. The transaction management system 2 delivers, for example, various data to be transacted, to each transacting institution or user terminal. The destination of delivery may be, for example, a non-profit incorporated association or the like, and, in addition to CSV format, for example, the delivery location may be converted into a predetermined data format desired by the transaction management system 2, and delivered.

<Authentication Information Assignment System 5>

The authentication information assignment system 5 certifies that, for example, an institution is a public or authorized authenticated institution, and that a health check institution or medical care institution is a certified institution. The authentication information assignment system 5 sends the result of authentication to each health check institution, medical care institution, and the like.

The authentication information assignment system 5 assigns authentication information, which indicates that the health check institution (for example, hospital), the testing institution and the like are institutions that have undergone authentication. Furthermore, the examinee and the user, who is the parties to the highly reliable data (medical examination/medical care data etc.) and the user are also assigned authentication information that indicates that they are authenticated. The result of authentication may be given in the form of predetermined authentication information, and, for example, an identification number or an identification mark that has been processed, designed or the like so as not to be tampered with may be used.

For example, once a health check institution is authenticated for the first time, the authentication information assignment system 5 may then authenticate the health check institution every few years—for example, every year, every two years, and so on. By doing so, it is possible to grant efficient authentication to each requested institution.

Although, for example, whether the source of the highly reliable data is a proper one is certified by the health check institution or an authenticator (a doctor, for example), and whether the authenticator has the authority for authentication is confirmed by the authentication information assignment system 5.

For example, in order to ensure the presence of authority for authentication on a real-time basis, the authentication information assignment system 5 checks whether or not the authority for authentication is granted, via a medical staff database (not shown), based on identification information for identifying various health check institutions and the authenticators of health check institutions (for example, medical license, medical license number, etc.), and based on registered information held by related parties (for example, administrative institutions such as the Ministry of Health, or licensed/registered institutions such as health centers).

The authentication information assignment system 5 checks whether or not an authenticator is a legitimate authenticator or whether or not a health check institution is a legitimate health check institution, depending on the presence or absence of the authority for authentication, with reference to the medical staff database. The authentication information assignment system 5 performs authentication check based on these check results and a series of these authentication-related processes.

There may be a plurality of authentication information assignment systems 5, in addition to the health check institution authentication, for example. The authentication information assignment system 5 may authenticate functions and services provided in other fields, businesses, and health check institutions, in addition to the authentication of health check institutions, for example.

The authentication information assignment system 5 may allow each authentication information assignment institution to perform authentication individually or all together. The authentication information assignment system 5 is authenticated by, for example, a licensed person for authentication, an authentication staff member or the like, based on various information recorded in the authentication information assignment database 5b, via the authentication information assignment device 5a.

The authentication information assignment device 5a provided in the authentication information assignment system 5 has, for example, the same configuration as the configuration of the examinee terminal 4 shown in FIG. 2 described above, or the configuration of a system comprised of microservices or a cloud system (not shown), and may include, for example, a housing (not shown), a CPU, a ROM, a RAM, a storage unit, and a plurality of I/Fs. Each configuration may be connected by an internal bus.

In addition to health check institutions, the authentication information assignment system 5 may authenticate, for example, information related to examinees who have taken examinations in health check institutions, and the users. In order to authenticate the health check institution system 3 from the health check institution device 3a, the authentication information assignment system 5 may receive, for example, various information related to the health check institution system 3, and authenticate the health check institution system 3, the examinee, or the user, based on various information stored in the database 5b, via the authentication information assignment device 5a.

To be more specific, the authentication information assignment device 5a authenticates the examinee and the user, based on health check information and personal information, from the health check institution, for specifying the examinee and the examinee's health check result, registration information for indicating that the user is an authenticated (legitimate) user, and the like.

The authentication information assignment device 5a includes an authentication information assignment database 5b, and has tables related to health check institutions (health check institution system 3), examinees (examinee terminals 4), and users (user terminals 6). Each table stores IDs for identifying each, and stores various information in association with these IDs.

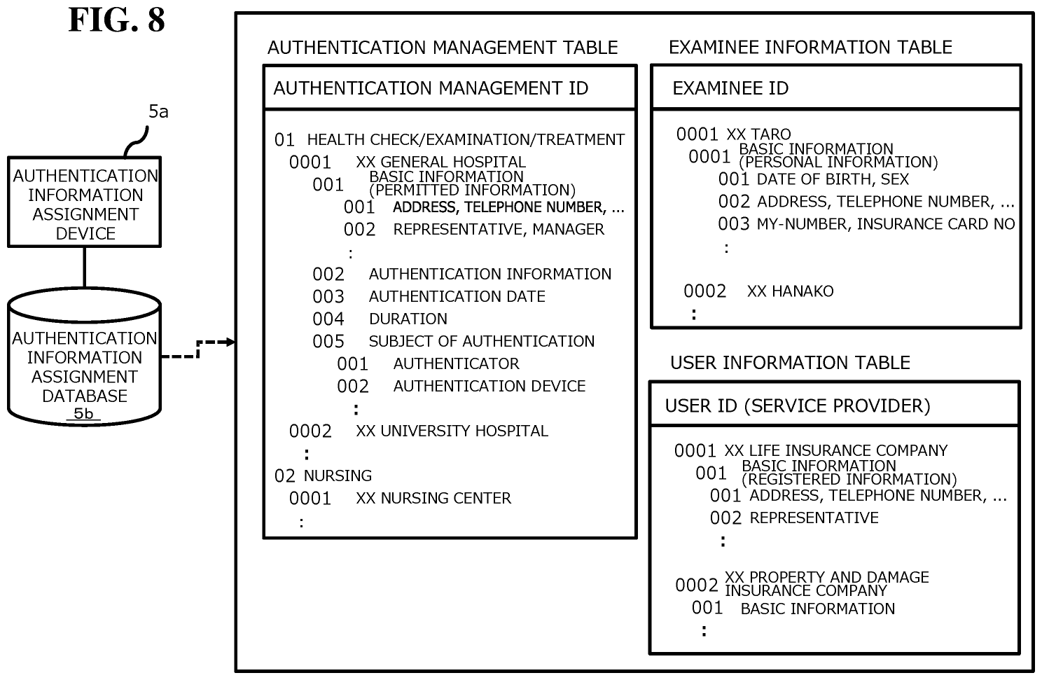

The configuration of the authentication information assignment database 5b will be described later with reference to FIG. 8. The authentication information assignment database 5b transmits, for example, the result of authentication in the authentication information assignment device 5a, after the authentication, to the health check institution system 3 that has requested authentication. After the authentication, the authentication information assignment device 5a transmits the authentication result to each health check institution, examinee, and user that have requested authentication.

The authentication information assignment device 5a may set the duration of validity of authentication information, and, in that case, for example, the duration of validity of authentication information is recorded as one year. The duration of validity, the date of expiration and so forth of authentication information may be set depending on, for example, the number of years and the scale of establishment of the health check institution, the number of examinees in the health check institution, and so on, and how to set it is unspecified.

Also, the authentication information assignment system 5 may take, from the transaction management system 2, the examinee terminal 4, the user terminal 6, the highly reliable data storage system 7 and the like, inquiries regarding the authentication of the health check institution such as whether or not the health check institution is an authenticated health check institution, whether or not the authentication information is effective, and so forth.

The authentication information assignment system 5 may reference various information stored in the authentication information assignment database 5b, from the information related to the health check institution (for example, health check institution name, authentication ID, etc.), received as input via each system or each device, and answer each inquiry from the transaction management system 2, the examinee terminal 4, or the user terminal 6, including whether or not the health check institution is authenticated. Also, various authentication results in the authentication information assignment system 5 are stored in the highly reliable data storage database 7b, via the highly reliable data storage device 7a of the highly reliable data storage system 7.

<User Terminal 6>

The user terminal 6 is used by, for example, a service provider that provides a new service or a new product to an examinee. The user submits a request for the use of highly reliable data (medical examination/medical care data, etc.) that he/she wants to use, to the examinee of the highly reliable data, via the user terminal 6, and uses the highly reliable data, according to the access rights granted by the examinee.

The user of highly reliable data may be, for example, a medical device manufacturer, a pharmaceutical manufacturer, a health service company, and the like. The prospective user submits a request for using the highly reliable data (medical examination/medical care data, etc.) that he/she wants to use, via the user terminal 6, and uses the data according to the access rights set by the examinee, via the transaction management system 2.

The user may be, for example, the examinee, or a representative delegated, entrusted or requested by the examinee. As a result, the representative can submit a request for the use of highly reliable data (medical examination/medical care data, etc.) that he/she wants to use, for example, via the user terminal 6 (or the representative's terminal, which is not shown), and use the data according to the access rights set by the examinee, via the transaction management system 2.

The user terminal 6 has the same configuration as the configuration of the examinee terminal 4, described above. For the user terminal 6, for example, an electronic device such as a personal computer (PC) may be used, and, besides, an electronic device such as a smartphone or a tablet terminal may be used. The user terminal 6 is configured as shown in FIG. 2, and, although not illustrated, like the configuration of the examinee terminal 4 shown in FIG. 2, includes a housing (not shown), a CPU, a ROM, a RAM, a storage unit, and a plurality of I/Fs. Each configuration is connected by an internal bus 108.

<Highly Reliable Data Storage System 7>

The highly reliable data storage system 7 stores, for example, highly reliable data (medical examination/medical care data, genetic data, etc.) from health check institutions (hospitals, etc.), testing institutions and the like, examinee consent data, various highly reliable data, various data or information such as correction data, various authentication information assignment information authenticated by the authentication information assignment system 5, and so forth, in the highly reliable data storage database 7b.

The highly reliable data storage system 7 transmits or acquires various highly reliable data via a public communication network or a distributed network 8. The highly reliable data storage system 7 stores these various data and information in the highly reliable data storage database 7b, by means of the highly reliable data storage device 7a. Note that the configuration of the highly reliable data storage database 7b will be described later.

The highly reliable data storage device 7a has, for example, the same configuration as the configuration of the examinee terminal 4 shown in FIG. 2 described above, or the configuration of a system comprised of microservices or a cloud system (not shown), and may include, for example, a housing (not shown), a CPU, a ROM, a RAM, a storage unit, and a plurality of I/Fs. Each configuration may be connected by an internal bus.

Based on the transaction in the transaction management system 2, the highly reliable data storage system 7 may provide part or all of the highly reliable data to the user terminal 6 based on, for example, the access rights set or granted by the examinee to the user terminal 6. Note that, these may be provided, for example, by artificial intelligence. Here, “artificial intelligence” may be based on any well-known artificial intelligence technology.

<Distributed Network 8>

The distributed network 8 connects between the transaction management system 2, the health check institution system 3, the examinee terminal 4, the authentication information assignment system 5, the user terminal 6, and the highly reliable data storage system 7, based on existing blockchain technology. The distributed network 8 connects, for example, the authentication of the health check institution related to the health check information of the examinee, the consent information of the examinees, the information related to the medical device, and, furthermore, information related to the use and/or utilization of highly reliable data, to the blockchains built in the distributed network 8, each as a block, and records these information.

The distributed network 8 may be connected with other networks (public communication networks), or, furthermore, may be connected with a plurality of blockchains built in a distributed network, in which transactions with other services are recorded, via the transaction management system 2. With which blockchains built in the distributed network transactions are carried out is unspecified.

(Example of Authentication Information Acquisition Operation)

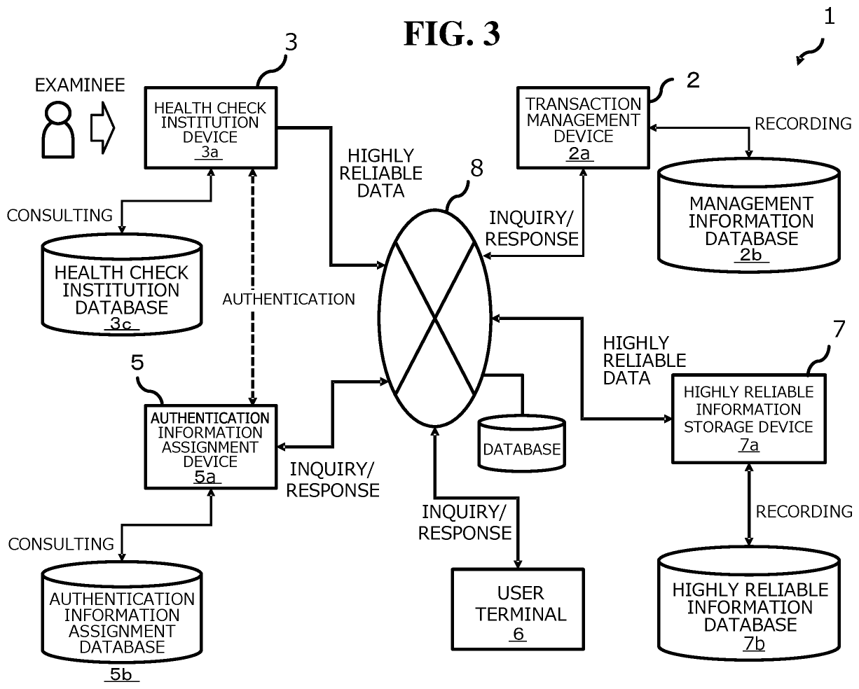

Here, an example of the operation of attaching authentication information to each institution in the health check institution system 3 and the authentication information assignment system 5 according to the embodiment will be described. FIG. 3 is a schematic diagram to show an example of acquisition of authentication information according to one embodiment.

As shown in FIG. 3, the health check institution device 3a of the health check institution system 3 requests the authentication information assignment system 5 to prove that its health check institution is an authenticated health check institution. The health check institution device 3a, for example, consults the health check institution database 3c for a predetermined authentication information assignment institution for applying authentication for, and acquires various application forms and data (not shown) necessary for authentication.

The health check institution device 3a consults the “health check institution information table” stored in the health check institution database 3c, acquires basic information registered regarding the health check institution, as necessary information (the year of establishment, the name of the hospital, the director, the address, the year of establishment, etc.), and transmits various information, including the basic information acquired, to a predetermined authentication information assignment system 5, via the blockchains built in the distributed network 8. The health check institution device 3a may transmit various information, including the basic information acquired, to the authentication information assignment system 5 via a network (for example, a public switched telephone network, etc.) apart from the distributed network 8, for example.

The authentication information assignment device 5a authenticates the health check institution with reference to various authentication group information and the like, stored in the authentication information assignment database 5b. The authentication information assignment system 5 reviews various application forms and data submitted by the health check institution system 3 and the like, based on various information (not shown) of public institutions for authenticating health check institutions, or third-party institutions, stored in the authentication information assignment database 5b. If it is judged that there is no problem with the health check institution or the health check institution system 3, authentication information is generated, and the generated authentication information is transmitted to the authentication information assignment system 5 for which the authentication that requested the authentication.

The health check institution system 3 receives the authentication information from the authentication information assignment system 5. The health check institution system 3 stores the received authentication information in the health check institution database 3c. After that, the health check institution system 3 performs medical examinations, via each health check institution device 3a, for example, with a predetermined examination menu or the like predetermined for each examinee.

In the health check institution system 3, for medical examinations for examinees, for example, various health check institution devices 3a and the like are operated by doctors and staff in charge at the health check institutions, and the medical device 3b and the like are operated to perform various medical examinations. The health check institution device 3a outputs each examined examinee's health check result, via the medical device 3b, as health check information. After confirming the health check results, the health check institution device 3a assigns authentication information which indicates that, for example, a health check result has been examined by a certified health check institution, to the health check information.

The health check institution device 3a stores various information such as various health check results examined at the health check institution device 3a, authentication information to indicate that the health check institution has been authenticated by the authentication information assignment institution, device information of the medical device 3b, and correction information for the health check information, in the examinee information table of the health check institution database 3c, which will be described later, as examinee information for identifying the examinees.

The health check institution device 3a generates the highly reliable data to be utilized by the examinees. The highly reliable data includes the health check results of the examinees, the correction information related to the examination results, and the authentication information indicating that the authentication has been rendered by the authentication information assignment device 5a.

The highly reliable data includes, at least, authenticator information related to an authenticator performing the authentication through the authentication means, authentication information including date/time information to show the date and time of the authentication, consent information of the examinee, health check institution information for identifying the health check institution where the examinee takes the health check, medical device information for acquiring the health check result data, assignment information related to correction of standardization, use information related to use of the highly reliable data, attribute-specific reference value comparative standard deviation data provided per examinee attribute, aging transition data of the examinee, or future predicting data of the examinee.

The health check institution device 3a acquires assignment information. The assignment information includes, at least, standard deviation correction data for the health check result data, attribute-specific reference value comparative standard deviation data, transition data, analysis data, device information or common language that serves as a standard for standardization, standardization model data, and token information for use in transactions.

By this means, even if there are differences in health check information such as the health check institution where the examination takes place, the medical device used for the examination, and the numerical values or graphs of the health check results of the examinees, the health check institution device 3a can still standardize these health check information, so that it becomes be possible to use these health check information on a wide scale, to design and propose various services. Furthermore, it becomes possible to facilitate the development of services and products that are more suitable for both examinees and users, and the provision of products, services and financial services with additionally-high transaction values.

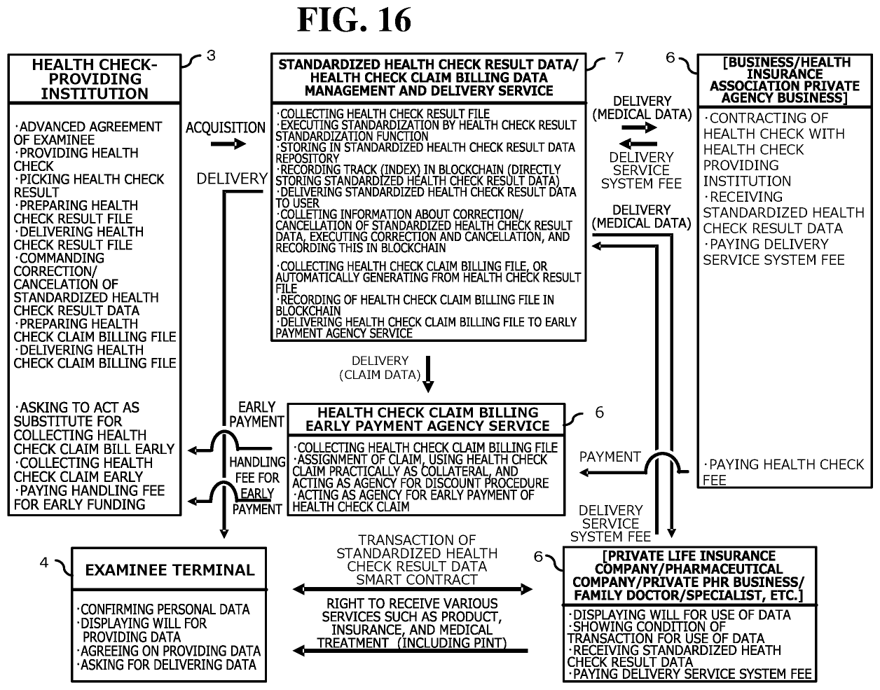

Now, with reference to FIG. 16, an example of delivery of standardized health check result data according to one embodiment will be described.

FIG. 16 is a schematic diagram to show an example of delivery of standardized health check result data according to one embodiment. The highly reliable data storage system 7 acquires the health check data (which may also be health check information, highly reliable data, examination data, health check result files, etc.) of the examinees who have taken a health check at the health check institution and whose highly reliable data stored in the blockchains has been acquired.

The highly reliable data storage system 7 acquires highly reliable data, to which the examinee has given consent, from the blockchain, standardizes the acquired highly reliable data based on the consented examinee's authentication information, and generates standardized health check result data, which is standardized.

The highly reliable data storage system 7 judges the need for standardizing the health check data of the examinees acquired from the health check institution system 3, and standardizes the health check data according to the result of judgement. After that, the highly reliable data storage system 7 transmits the standardized health check result data to the insurer, such as a health insurance association.

The highly reliable data storage system 7 varies depending on, for example, various measuring instruments, machines, or development languages used when the examinees take medical examinations, or used in the hospitals or institutions where the examinees take medical examinations, and might also vary depending on, for example, the programs for analysis, various settings, the environment of examination (for example, room temperature, humidity, weather, physical condition of the examinee on the day, etc.), so that standardization into user-friendly standardized health check result data is carried out.

The highly reliable data storage system 7 acquires, for example, the health check data (health check result data, health check result files, etc.) acquired through the software (for example, standardization software) for standardizing the differences in health check data, standardizes the acquired health check data, and generates the standardized health check result data.

The highly reliable data storage system 7 may include standardization software for standardizing the acquired health check data, and record the standardized health check data in the transaction management database 2b of the transaction management system 2, for example. When the standardized health check result data is to be corrected, each health check institution system 3 commands the highly reliable data storage system 7 to correct or cancel the standardized health check result data.

The highly reliable data storage system 7 attaches, for example, flag data to indicate that standardization has been performed, to the standardized health check result data. The highly reliable data storage system 7 may attach the flag data to the standardized health check result data, the health check data, or the collected highly reliable data.

The highly reliable data storage system 7 records, for example, the standardized health check result data, as highly reliable data, together with flag data to indicate the standardized health check result data and the completion of standardization, data of examinees or the consent of examinees, and authentication data to indicate that that the health check data is legitimated by the health check institution that device 3a, in the blocks connected by the blockchains built in the distributed network 8.

The highly reliable data storage system 7 may standardize the standardized health check result data based on, for example, a standard health check model or the like in a system (not shown) of a non-profit corporation, and store the standardized health check result data in a repository for standardized health check result data. Furthermore, in a system in a non-profit corporation, for example, time stamps are attached, as track data, to the data related to all exchanges related to delivery, and the data is recorded in the blocks connected by the blockchains built in the distributed network 8, in chronological order, based on the time stamps attached.

Also, the system in the non-profit incorporated association may transmit the standardized health check result data, to the health insurance association (or its agent institution), which is the insurer, which is the contracting subject of the health check institution, and/or to a third party or a third party institution that the person (examinee) approves, in, for example, CSV data format (or XML data format). At this point, the non-profit incorporated association may collect a predetermined system use fee or the like from the third party or the third party institution, and pay the operator of the transaction management system 2 in compensation (for example, pay the use fee for the first settlement, the digitization fee for the second settlement, etc.).

The standardized health check result data may be stored (for a backup) as, for example, life-long received data (health check data) based on individual consent, per examinee, in the database of the system in the non-profit corporation (for example, the highly reliable data storage database 7b or a life-long information database (not shown)). The life-long examination data of each examinee backed up in the database in the system of the non-profit corporation may be stored in, for example, blocks (track data) connected by a life-long blockchain built in the distributed network 8 (in which information of the examinees is recorded in association with my-numbers). Consequently, the required track data may be extracted from the blocks recorded in the life-long blockchain, based on the my-numbers of the examinees.

The track data is data transacted with, for example, a third party, a third party institution, or the like. As for the track data to be transacted, for example, the track data required by a third party or a third party institution may be received by confirming the examinee's will again, regarding the track data through the examinee terminal 4.

The health check implementation institution (health check institution system 3) extracts, for example, the agreed health check results of examinees from the database of each institution, and transmits the extracted health check results, to the standardized health check result data delivery service institution (highly reliable data storage system 7), as health check result files. The health check implementation institution receives the health check result file based on the service use contract with the standardized health check result data delivery service institution (device), and performs various standardizations and delivery to users.

Also, the standardized health check result data delivery service institution device standardizes the received health check result data. The standardized health check result data delivery service institution device extracts the standardized health check result data from the received health check result files, and delivers the standardized health check result data to the users who have requested the use (for example, health promotion business operator, etc.).

Also, the standardized health check result data delivery service institution device has each function of payment provisional agent and testing contract. The standardized health check result data delivery service institution device includes, furthermore, a health check standard data DB, and records the collected health check result file, health check result data, standardized health check result data, and the like. Furthermore, the standardized health check result data delivery service institution device records various transaction-related information as track data, in blocks connected by the blockchains built in the distributed network 8.

Also, the standardized health check result data delivery service institution device, various standardized data (track data), and, furthermore, the health check result file from the standardized health check result data delivery service are exchanged with a health promotion business operator such as, for example, a health insurance association, a private agent institution, a private PHR company, a company, and so forth, and recorded in the blocks connected by the blockchains built in the distributed network 8.

Also, with the standardized health check result data delivery service institution device, the connected verification database checks the credibility of the health check implementation institution and the health promotion business operator, as well as the standardized health check result data, the details of the contract, and the subject of transactions. The standardized health check result data delivery service institution device may use identification information such as the health check data ID, the examinee ID, and the user ID from the examinee terminal 4 or the user terminal 6, to search for various types of information stored in the health check standard database.

The health promotion business operator device (user terminal 6) receives the standardized health check result data from the standardized health check result data delivery service institution device, and settles the payment process. The health promotion business operator device may settle the payment to each of the standardized health check result data delivery service institution and the health check implementation institution, based on various conditions such as the type of received standardized health check result data, the amount of data, and the person taking a health check.

Then, the health check institution device 3a records the generated highly reliable data for each examinee, in the blocks connected by the blockchains built in the distributed network 8. The highly reliable data generated by the health check institution device 3a includes recorded data that corresponds to the authentication information assignment institution, such as when the authentication information assignment institution did the authentication, based on the authentication information transmitted from the authentication information assignment device 5a.

Furthermore, the health check institution device 3a may convert the conventional paper information into PDF and store the necessary information in a reading format as recorded data. The health check institution device 3a may, for example, utilize general AI cognitive technology or the like, to convert examination data and the like into CVS data.

Also, the health check institution device 3a monitors the amount of processing handled by AI, for example, and if, as a result of this, it is judged that characters and data that cannot be processed by AI are scattered in the target business range or processing range, for example, standard procedures with other systems where various data such as examinations data is manually captured may be provided to each health check institution. By this means, the health check institution device 3a can, for example, have other systems collectively perform processes even when the processes concentrate on AI in the health check institution or the testing institution, and a highly-loaded condition arises. By this means, for example, it becomes possible to collect technical use fees such as AI or business consignment fees.

Also, the transaction management system 2 acquires various highly reliable data, related to a plurality of examinees and generated in various health check institution systems 3, via the blockchains built in the distributed network 8. The transaction management system 2 records the acquired various highly reliable data in the transaction management information database 2b.

The transaction management system 2 performs transactions of highly reliable data with the examinee terminal 4 and the user terminal 6, via the distributed network 8. The transaction management system 2, the examinee terminal 4, or the user terminal 6 may, for example, send an inquiry for the authentication information, contained in the highly reliable data, to the information assignment system 5, to confirm the credibility of the examinee's highly reliable data and the health check institution.

In response to the confirmation of the authentication information from the transaction management system 2, the examinee terminal 4 or the user terminal 6, the authentication information assignment system 5 identifies the transaction management system 2 or the user terminal 6, where confirmation has been requested, with reference to each table stored in the authentication information assignment database 5b. The authentication information assignment system 5 may return the result of the inquiry for authentication information, to the terminal or the like for which the inquiry was made, according to the result of the reference.

(Example of Operation for Granting Access Rights)

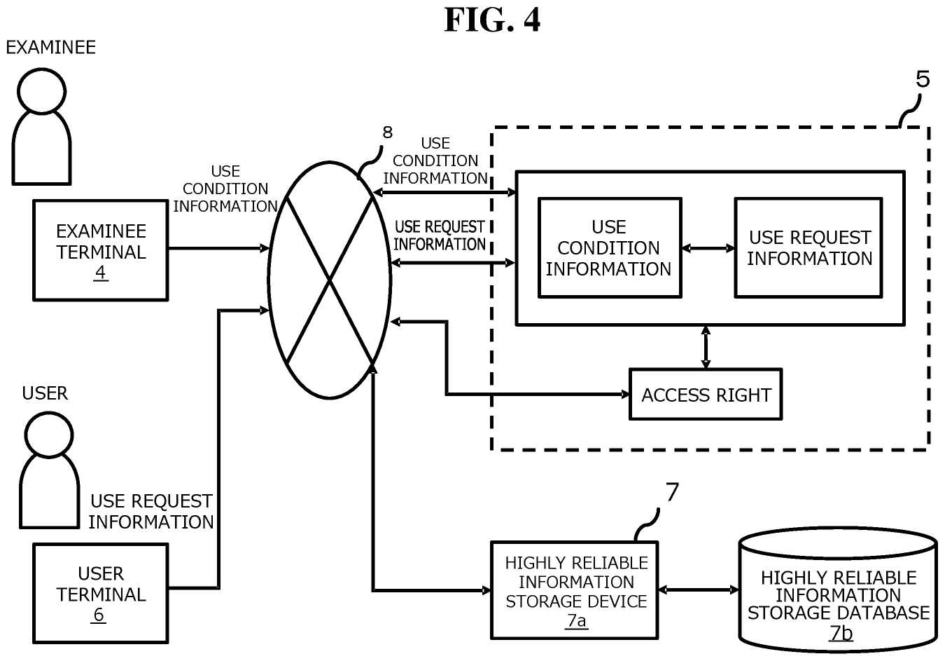

Here, an example of the operation for granting access rights to the highly reliable data of desired examinees on the examinee terminal 4, the authentication information assignment system 5, and the user terminal 6 according to one embodiment will be described. FIG. 4 is a schematic diagram to show an example of granting access rights according to one embodiment.

First, regarding the highly reliable data that the examinee wants the user and the like to utilize, among the examinee's highly reliable data examined at the health check institution, the examinee determines, in advance, various information regarding the use of highly reliable data such as, for example, whether the whole or part of the health check result is used, the period the data is available for use, the price or points of use, and so forth, through the examinee terminal 4. The examinee records these conditions of use, use condition information included in highly reliable data, and so forth, in association with the highly reliable data, in the blocks connected with the blockchains built in the distributed network 8, via the examinee terminal 4.

Also, the examinee may set the use condition information so as to acquire, for example, only the necessary highly reliable data related to the examinee himself/herself through the examinee terminal 4. By this means, the examinee can acquire and provide only the data specified by the examinee via the examinee terminal 4.

As for the conditions of use, for example, the image of the examinee's face image, his/her voice data, fingerprint data, genetic data, or environmental data for specifying the location (for example, communication status at the time of setting, BODY DATA such as the device's radio wave information, etc.) for use for authentication in transactions of highly reliable data, may be set as the examinee's authentication data, for the examinee terminal 4, the transaction management system 2, and other related devices and systems. By this means, it is possible to increase the strength of authentication in transactions of highly reliable data.

The use condition information including these setting-related information may be associated with highly reliable data, and recorded in the blocks connected by the blockchains built in the distributed network 8. The highly reliable data may be arranged so that highly reliable data, to which hash data is attached, is recorded in the blocks connected by the blockchains built in the distributed network 8.

Also, for the conditions of use, for example, the value of highly reliable data may be set. The value of highly reliable data is, for example, the right to receive money, coins, or goods, the right to receive services such as new insurance products and various health services, and the right to receive benefits provided from the users of highly reliable data. These values are registered via the examinee's own examinee terminal 4 or on my-page on the Web.

The registered values can be referenced through the examinee terminal 4 or on my-page, and, furthermore, the registered values—for example, records of value transactions, information such as the balance after transactions—are recorded on the blockchains. The value-related information recorded on the blockchains may be recorded on the blockchains so that, for example, when the right to a value is fulfilled, the corresponding value record disappears, and all the records of this are recorded on the blockchains (for example, acquisition of additional highly reliable data, delivery record, transaction record, etc.).

The transaction management device 2a of the transaction management system 2 records various use condition information for highly reliable data of the examinees, via the blockchains built in the distributed network 8, in the “Examinee management table” of the transaction management information database 2b, in association with the examinee IDs of the examinees. The highly reliable data of the examinees is later referenced by the users of the user terminal 6, via the blockchains built in the distributed network 8.

The user is, for example, a user who has been authenticated by the authentication information assignment institution, and can refer to highly reliable data permitted to be used by the examinee. If, through the user terminal 6, there is an examinee's highly reliable data the user wants to use, the user generates a use request for the use of that highly reliable data as use request information, and the user terminal 6 applies for use, to the transaction management system 2. The highly reliable data is stored in the highly reliable data storage database 7b of the highly reliable data storage system 7, and provided based on the setting of access rights.

In the transaction management information database 2b, the use request information generated in the user terminal 6 is stored as a “user information table”. In the “user information table”, for example, in addition to information related to the user, various request information such as the type and range of highly reliable data to be used and the period to be used may be registered as use request information. The use request information may further include, for example, the price paid or points given by the user for highly reliable data.

Also, the transaction management device 2a allows the examinee terminal 4 to reference “001 Examinee ID, 005 Conditions of use,” which is registered in advance with the “Examinee management table” of the transaction management information database 2b, a comparison with the price offered by the user terminal 6 is made. If the conditions of use set by the examinee are satisfied according to the result of each price's comparison, the transaction management device 2a establishes transaction of the highly reliable data requested for use with the user, and the information of this establishment may be recorded in blocks connected by a series of blockchains.

The transaction management device 2a consults the transaction management information database 2b, and grants access rights to the highly reliable data that the user wants to use, based on various information related to the conditions of use of highly reliable data of examinees, recorded in the “Examinee management table”, and the use request information received by the user terminal 6. The transaction management device 2a adds the history of recorded contents, to the blocks of the blockchains built in the distributed network 8, including the acquisition of use condition information, the use request information received, and information of the history of granting of access rights, and so forth. Each examinee may set the granting of access rights by the transaction management system 2, for example, on his/her my-page, via the examinee terminal 4.

As for the setting of access rights by the examinee terminal 4, if the conditions of use are set by the examinee in advance, the access rights are granted based on the recorded conditions of use. With the transaction management device 2a, if no conditions of use are set for highly reliable data, or when there is a new request for use, for example, a notice of confirmation of use condition information set by the examinee on the examinee terminal 4 may be transmitted from the transaction management device 2a.

If there is a change in the conditions of use that are already set and registered in the transaction management information database 2b of the transaction management system 2, the examinee terminal 4 has that change reflected in the original conditions of use, and, for example, records the changed content and the history, such as the date of change, as “001 Examinee ID, 007 Update date information”.

Processes such as the acquisition, authentication, or transaction of highly reliable data described above are controlled by, for example, artificial intelligence. For control by artificial intelligence, for example, deep learning may be used for machine learning. The various databases described above may further include, for example, a reference database, which may be built with a neural network. In the reference database, for example, the first association may be represented by a hidden layer and a weight variable.

In the highly reliable data transaction system 1, for example, a reference database built on machine learning can be used. In this case, the complex relationship between past or known target information concerning a plurality of types of data and reference information can be represented by the first association, with high accuracy. By this means, it is possible to improve the accuracy of acquisition and judgment results.

Also, when the reference database is comprised of, for example, a neural network, the first to third associations, and the similarity may indicate weight variables that are associated with selected past or known evaluation information, and be judged based on these associations.

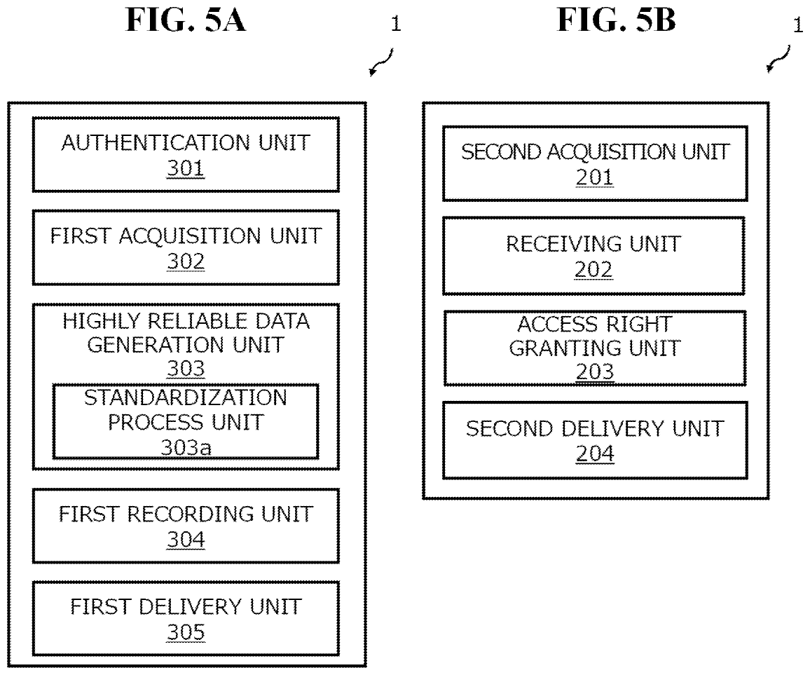

FIG. 5A is a schematic diagram to show examples of functions of the highly reliable data transaction system 1 according to one embodiment. The highly reliable data transaction system 1 includes, at least, an authentication unit 301, a first acquisition unit 302, a highly reliable data generation unit 303, a standardization process unit 303a, a first recording unit 304, and a first delivery unit 305. Note that the functions shown in FIG. 5A are configured the same as on the examinee terminal 4 shown in FIG. 2, and, although not illustrated, for example, an electronic device such as a personal computer (PC) may be used, or a configuration such as a system comprised of microservices or a cloud system may be used.

<Authentication Unit 301>

The authentication unit 301 authenticates the health check institution that acquires the health check information acquired in the first acquisition unit 302. The authentication unit 301 authenticates the health check institution system 3 by transmitting and receiving health check institution information to and from the authentication information assignment device 5a of the authentication information assignment system 5 constituting the highly reliable data transaction system 1. The authentication unit 301 transmits various basic information of the health check institution system 3 transmitted from the health check institution device 3a, including, for example, license information of the health check institution, health check institution information such as the year of establishment, the name of the hospital, the director, the address of the health check institution, and the year of establishment, authentication information that is authenticated based on the health check institution information, medical device information, and so forth, to the health check institution system 3.

Note that, if authentication information is already acquired in the health check institution system 3, the health check institution device 3a may acquire the authentication information stored in the health check institution database 3c, without sending an authentication request to the authentication information assignment system 5, and attach the authentication information to the health check information received in the health check institution device 3a or the like. The health check institution device 3a acquires the authentication information and the like via the I/F 105, and, furthermore, acquires various information input from the input part 110 via the I/F 105.

<First Acquisition Unit 302>

The first acquisition unit 302 acquires health check information related to the medical care which the examinee took. The first acquisition unit 302 acquires various health check information of the examinee through the medical device 3b, in the health check institution device 3a of the health check institution system 3 constituting the highly reliable data transaction system 1. The first acquisition unit 302 acquires the health check information of the examinee measured by the medical device 3b via an I/F, and, furthermore, acquires various information received as input from the input part via the I/F.

The health check information of the examinee acquired in the first acquisition unit 302 is appropriately selected depending on, for example, each examinee's body condition and state, gender, age, etc., in addition to general health check examination menu. The health check information may be, for example, information related to the health check results of the examinee measured by a health check institution, a medical care institution, etc., and may include text information of doctors' opinions, image data of affected body parts photographed, or examination data such as the results of past health examinations conducted at other health check institutions and medical care institutions.

The first acquisition unit 302 stores, for example, various information measured at other health check institutions, or measured by the examinees at other places or at home, and recorded on a cloud, via the distributed network 8 or a medium such as a portable memory. Note that the file format of various information acquired in the first acquisition unit 302 is unspecified, and, for example, the first acquisition unit 302 may carry out conversion an arbitrary file format.

<Highly Reliable Data Generation Unit 303>

The highly reliable data generation unit 303 generates highly reliable data, related to an examinee, based on authentication information authenticated in the authentication unit 301 and the examinee's health check information. The highly reliable data generation unit 303 generates corrected highly reliable data related to the examinee, based on the authentication information authenticated in the authentication unit 301, and the examinee's corrected health check information.

Also, as other assignment information of the health check information, which is the result of the examination, the highly reliable data generation unit 303 may include, for example, personal identification information for identifying the individual who took the examination, consent form information related to the use of the health check information, authentication information of the authentication information assignment institution, information related to the date of authentication, and information related to the health check institution (or health check institution) the examination was taken, information related to the medical device used in the examination, information related to the date and time of the examination, assignment information for the health check information, the medical device, or the standardization, and standard deviation correction data for the health check information.

Also, the highly reliable data generation unit 303 consults the management information database 2b or the highly reliable information database based on the generated highly reliable data of the examinee, extract, for example, various information of the examinee as information related to the examinee of the highly reliable information, and generate highly reliable data of the examinee.

<Standardization Process Unit 303a>

The standardization process unit 303a acquires the highly reliable data, generated by the highly reliable data generation unit 302, from the blocks recorded by the first recording unit 304 and connected by the blockchains built in the distributed network 8, based on the consent information acquired in the second acquisition unit 201, and standardizes the acquired highly reliable data.

In the event the protocol is standardized, the standardization process unit 303a standardize the name, the year and date of birth, health check items, health check results and other data items, based on the examinee information related to the examinee who took the health check, and information related to the medical device used for the health check in each health check institution.

The standardization process unit 303a standardizes, for example, the protocol, the terms/code, or the format. In the event the protocol is standardized, the standardization process unit 303a standardizes, for example, the data items to exchange (for example, the name, the year and date of birth, the health check items, the health check results, etc.), the rule for describing each data item (for example, the name is to be written in order of the surname, the first name and the middle name), and the exchange message format (the items to include in the request information, the items to include in the result information, etc.). In the event the terms/code are standardized, the standardization process unit 303a standardizes the terms (for example, unifies “GAMMA-GTP” and “γ-GTP”), the codes (coordination of medical codes and article codes), and the like. Furthermore, in the event the format is standardized, the standardization process unit 303a standardizes the image format (DICOM format, JPEG, BMP, etc.), the waveform format (electrocardiogram, electrosurgical meter, etc.) and the like.

For example, for the standardization that is needed for the sharing/coordinating of health check results and medical information so as to exchange health check result data, the standardization process unit 303a acquires the highly reliable data generated in the highly reliable data generation unit 302, from the blocks recorded in the first recording unit 304 and connected by the blockchains built in the distributed network 8, based on the consent information acquired in the second acquisition unit 201, and standardizes the acquired highly reliable data.

<First Recording Unit 304>

The first recording unit 304 delivers based on various transaction information via the transaction management device 2 of the transaction management system 2. The first recording unit 304 stores part or all of the highly reliable data, divided into blocks, in the highly reliable data storage database 7b for storing highly reliable data, the blockchain 8 built in the distributed network, or in the database of the blockchain 8 built in the distributed network.

Apart from the highly reliable data storage database 7b for storing highly reliable data and the blockchain 8 built in the distributed network, the first recording unit 304 may record part or all of the highly reliable data, distributed over the blocks connected by the blockchains built on the examinee terminal 4. The recording of each data in the first recording unit 304 will be described later.

<Second Storage Unit>

The highly reliable data transaction system 1 includes a second storage unit (not shown). The second storage unit stores the health check result data standardized by the standardization process unit 303a, for standardizing part or all of the health check result data acquired in the first acquisition unit 302, as health check result data, in the reliable data storage database 7b or in the blocks connected by the blockchains built in the distributed network 8.

<Third Storage Unit>

The highly reliable data transaction system 1 includes a third storage unit (not shown). Based on the user delivery of part or all of the highly reliable data by the first to third delivery units described above, the third storage unit acquires track information and history information related to the result of delivery. The third storage unit records the acquired track information and history information in the blocks connected by the blockchains built in the distributed network 8.

<First Delivery Unit 305>

When the first delivery unit 305 acquires transaction information related to the acquisition of part or all of the highly reliable data recorded by the first recording unit 304, the first delivery unit 305 acquires part or all of the highly reliable data, depending on the transaction information.

Regarding the acquisition of highly reliable data, the first delivery unit 305 identifies the transaction information (for example, use, payment, price, etc.) between the examinee and the health check institution, and between the testing institution and the transaction management institution, and, depending on the details identified, identifies part (for example, index information, storage location information, abstract, bibliographic information, etc.) and all (health check result data, health check result file, etc.) of the highly reliable data delivered, specifies the users (distributors) to deliver the highly reliable data, and delivers the highly reliable data to the user terminals 6 associated with the specified users.

FIG. 5B is a schematic diagram to show examples of functions of the transaction management device 2a of the transaction management system 2 constituting the highly reliable data transaction system 1 according to one embodiment. The transaction management device 2a includes at least a second acquisition unit 201, a receiving unit 202, an access right granting unit 203, and a second delivery unit 204. Note that the functions shown in FIG. 5B are configured the same as on the user terminal 6 shown in FIG. 2, and, although not illustrated, for example, an electronic device such as a personal computer (PC) may be used, or a configuration such as a system comprised of microservices or a cloud system may be used.

The transaction management device 2a includes, for example, a housing (not shown), a CPU (Central Processing Unit), a ROM (Read Only Memory), a RAM (Random Access Memory), a storage unit, and a plurality of I/Fs, where each configuration is connected by an internal bus. The CPU controls the whole of the transaction management device 2a. The ROM stores operation codes for the CPU. The RAM is the task area for use when the CPU operates. The storage unit stores various information such as management information related to the examinees and users, in addition to the transaction-related information required for transacting blockchains. As for the storage unit, for example, a data storage device such as an SSD (Solid State Drive), a semi-conductor memory and the like may be used, in addition to an HDD (Hard Disk Drive).

The CPU is implemented by running programs stored in the storage unit and elsewhere, by using the RAM for the task area. Note that, for example, the transaction management device 2a may include a GPU (Graphics Processing Unit), which is not shown. Having a GPU enables faster calculation processing than usual.

<Second Acquisition Unit 201>

The second acquisition unit 201 provides use condition information related to the conditions of use of highly reliable data, via the examinee terminal 4 of the examinee. Also, the second acquisition unit 201 acquires consent information related to the transactions of highly reliable data from the examinee. The second acquisition unit 201 acquires the use condition information (chargeable data, prices, contract conditions, tokens, etc.) related to the conditions of use of highly reliable data of the examinee, from his/her examinee terminal 4, in the transaction management device 2a of the transaction management system 2, and stores this in the transaction management information database 2b. The use condition information input from the examinee terminal 4 is recorded in the blocks connected by the blockchains built in the distributed network 8. The user of the blockchains built in this distributed network 8 can use his/her user terminal 6, and consult the use condition information of the examinee's highly reliable data indicated by the examinee. The transaction management system 2 can receive use request information of highly reliable data from the user terminal 6.

<Receiving Unit 202>

The receiving unit 202 receives the use request information and collects the request from the user terminal 6 wanting to use the highly reliable data. The use request information may include, for example, information such as the type or category, the number of people, the duration and so forth, in addition to information related to the examinee of the highly reliable data that is wanted for use. Furthermore, upon use, the price may be presented in advance, so that it becomes possible to exchange highly reliable data for prices or points using smart contract technology. The user may include services that can be provided to the examinee, in the use request information.

The use request information received as input from the user terminal 6 is recorded in the blocks connected by the blockchains built in the distributed network 8. An examinee of a blockchain built in the distributed network 8 can consult the use request information indicated by the user using his/her own examinee terminal 4, and therefore can receive the use condition information for highly reliable data, from the examinee terminal 4.

<Access Right Granting Unit 203>

The access right granting unit 203 grants access rights to the highly reliable data recorded by the recording means, based on the use condition information for highly reliable data acquired in the second acquisition unit 201 and the use request information from the user terminal 6 received in the receiving unit 202. The highly reliable data, to which access rights are granted, may be, for example, stored in the transaction management information database 2b, and may be given an authentication code or the like that can be referenced, by the access right granting unit 203.

<Second Delivery Unit 204>

The second delivery unit 204 transacts part or all of the highly reliable data, to which access rights are granted in the access right granting unit 203, based on the use condition information, the use request information, and the access rights, and, furthermore, delivers part or all of highly reliable data to users (user terminal 6, alternative terminal, or business operators that require highly reliable data, etc.), depending on the result of transaction. The second delivery unit 204 executes a transaction of highly reliable data, standardized by the standardization process unit 303a, based on the use condition information acquired in the second acquisition unit 201.

The second delivery unit 204 delivers the highly reliable data based on various transaction information, via the transaction management device 2 of the transaction management system 2. The second delivery unit 204 performs transactions with, for example, a health check institution system 3, an examinee terminal 4, an authentication information assignment system 5, a user terminal 6, a highly reliable data storage system 7, and other systems (not shown). A plurality of transactions, payment, billing and so forth via the transaction management device 2 will be described later.

<Financial Data Recording Unit>

The highly reliable data transaction system 1 includes a financial data recording unit (not shown). The financial data recording unit records, for example, financial data related to the billing of the health check fee for the health check institution in the blocks connected by the blockchains built in the distributed network 8, related to the financing of highly reliable data.

<Claim Information Delivery Unit>

The highly reliable data transaction system 1 includes a claim information delivery unit (not shown). The claim information delivery unit gathers the same debtor's medical care claims, among the medical care claim information of the same health check institution, based on the health check institution information, the health check fee debtor information, the amounts of claims, and the due dates of payment, included in the financial data recorded by the financial data recording unit, and deliver these to the financial institution as one or more credit collateral information.

<Transaction Determining Unit>

The highly reliable data transaction system 1 includes a transaction determining unit (not shown). The transaction determining unit gathers the same debtor's medical care claims, among the medical care claim information of the same health check institution, based on the health check institution information, the health check fee debtor information, the amounts of claims, and the due dates of payment, and, gathering the credit collateral information, executes cloud lending based on one or more credit collateral information.

Depending on the delivery result of the credit collateral information by the claim information delivery means, the transaction determining unit selects a financial institution that can be funded by the health check institution. The transaction determining unit determines the assignment of claims, discounts on claims, or the staff in charge of claims, by the selected financial institution.

Also, the transaction determining unit gathers the same debtor's medical care claims, among the medical care claim information of the same health check institution, based on the health check institution information, the health check fee debtor information, the amount of claims, and the due date of payment. The transaction determining unit gathers the credit collateral information, and executes cloud lending based on one or more credit collateral information.

Based on the medical care fee included in the medical care/treatment fee information acquired, the transaction determining unit specifies the cases, in which the cost of treatment is determined to be high (for example, the difference from other treatment costs is hundreds to tens of millions of yen), as cases subject to cloud funding.

The transaction determining unit records information about authenticated institutions related to the identified target cases, highly reliable medical data related to medical care and treatment, and highly reliable medical financial data, in the blocks connected by the blockchains built in the distributed network 8.

The transaction determining unit gathers the same debtor's medical care claims, among the medical care claim information of the same health check institution, based on the health check institution information, the health check fee debtor information, the amounts of claims, and the due dates of payment, and, gathering the credit collateral information, executes cloud lending based on one or more credit collateral information.

<Right Acquisition Unit>

The highly reliable data transaction system 1 includes a right acquisition unit (not shown). The right acquisition unit consults the result of delivery of part or all of the highly reliable data by the first delivery unit 305, the second delivery unit 204, or the third delivery unit. Depending on the result of the consulting, the right acquisition unit determines information related to the right of the examinee and the transaction manager to receive intangible or tangible benefits. The right acquisition unit consults a plurality of information included in the information related to the right, and acquires information such as the proportion, share, price, and so forth, for the matching rights.

<Collecting Unit>

The highly reliable data transaction system 1 includes a collection unit (not shown). The receiving unit collects the corresponding benefits from the health check institution, the user, and so on, according to the rights acquired in the right acquisition unit.

<Third Delivery Unit>

Here, when transaction information related to the acquisition of part or all of the standardized health check result data, recorded by the second recording unit is recorded as highly reliable data in the highly reliable data storage database 7b for storing highly reliable data of the users from the first recording unit 304, in the blocks connected by the blockchains built in the distributed network 8, is acquired, the third delivery unit delivers part or all of the of the highly reliable data, depending on the transaction information.

<Evaluation Unit>

Evaluation results for highly reliable data are generated with reference to a reference database, in which a plurality of associations, corresponding to weight variables between a plurality of data included in past highly reliable data, acquired in advance, and a plurality of data included in the reference information linked with the past highly reliable data, are stored. The evaluation in the evaluation unit may provide for example, various information such as the result (text, numerical information, image, voice, etc.) of the highly reliable data of the examinee, information related to the examinee, information related to the highly reliable data, inquiries, requests, errors of each institution health check institution, and the like. Embodiments of evaluation will be described later.

<Notification Unit>

The notice unit extracts the providing information to be provided and the providing destination, based on the evaluation result in the evaluation unit, and reports the evaluation result and the provided information to the extracted providing destination. The information provided here may be, for example, reference information linked with the evaluation result, related information, caution information, improvement information, correction information, recommendation information, transaction information, or any other useful or valuable information. The provider may be, for example, a health check institution, an examinee, a user, a business operator, an institution related to the provided information, an association, an organization, a university, a research institution, or an individual. The notice period, time, format, frequency, and the number of times are unspecified.

<Updating Unit>

Based on the result of the notice from the notice unit, the updating unit acquires the highly reliable data recorded in the blocks connected by the blockchain by the second recording unit 204, and the corrected highly reliable data, updates the corresponding history, and records this information is in a new block connected by a blockchain built in the distributed network 8.

<Health Check Institution>

The health check institution may be a health check institution that provides health check data, a medical examination institution that provides medical examination data, a health check institution that provides various medical data, a testing institution that provides test information, a dental institution that provides dental data, a medicine institution that provides medicine data, and a gene institution that provides genetic data, and, in addition, various other institutions, for example, commercial or non-profit, and domestic and international institutions may be included.

<Highly Reliable Data>

Highly reliable data may include authenticator information related to the authenticator that performed authentication in the certification unit 301, authentication information, including date/time information to show the date and time of authentication, consent information of the examinees, health check institution information for identifying the health check institutions where the examinees took a medical examination, medical device for acquiring health check information, additional information related to the correction of standardization, use information related to the use of highly reliable data, attribute-specific reference value comparative standard deviation data, provided per examinee attribute, aging transition data of the examinees, storage location information for identifying the location where the health check result data is stored separately, my-number information for identifying the examinees, health insurance card information of the examinees, or future predicting data of the examinees, and, in addition, for example, information or data related to the examinee acquired in the health check institution.

<Assignment Information>

The assignment information is included in the highly reliable data generated by the highly reliable data generation unit 303. The assignment information may include, for example, standard deviation correction data for examination information, attribute-specific reference value comparative standard deviation data, transition data, analysis data, device information or common language that serves as a standard for standardization, or standard model data, as well as thresholds and parameters for use in various analyzes, developments, and transactions.

(Transaction Management Information Database 2b)

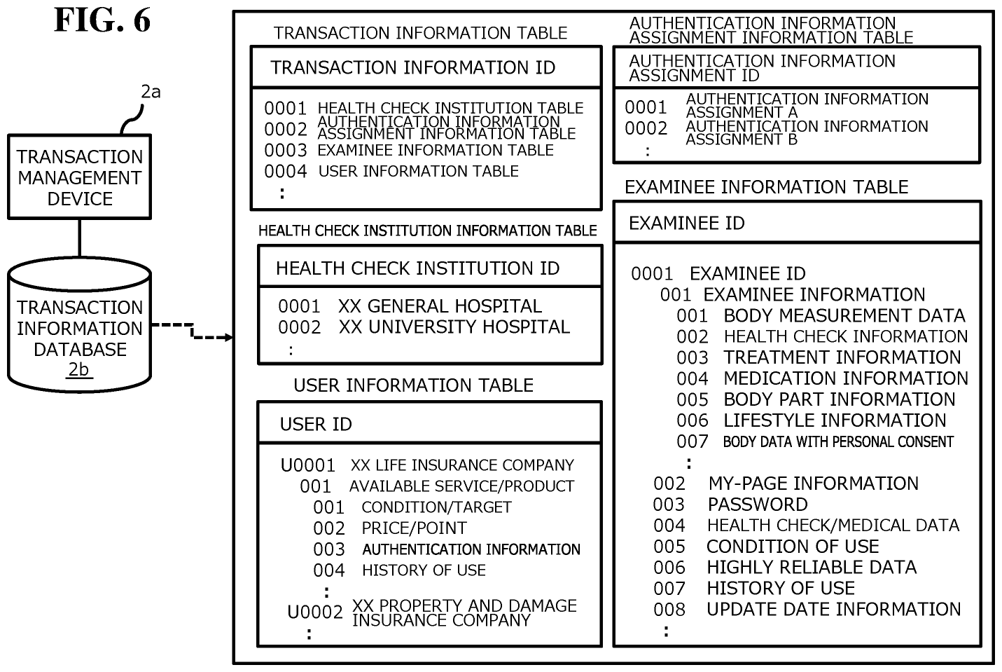

Next, with reference to FIG. 6, an example of the transaction management information database 2b connected to the transaction management device 2a according to the embodiment will be described.

As shown in FIG. 6, the transaction management information database 2b includes, at least, for example, “transaction information table”, “health check institution information table”, “authentication information assignment institution information table”, “examinee information table”, and “user information table”. a “transaction information table” to store information related to each institution, examinee, and user needed to manage transactions, a “health check institution information table” to identify health check institutions, an “authentication information assignment institution information table” to store information related to public institutions and the like that certify health check institutions, an “Examinee information table” to store various information related to examinees, and a “user information table” to store users' types of occupation and various information are stored in association with each other in the transaction management information database 2b.

In the “transaction information table”, for example, management information IDs that identify “0001 Health check institution information table” to store information of a list of health check institutions that can examine examinees or health check institutions and the like, “0002 Authentication information assignment institution information table” to store detailed data and information related to authentication information assignment institutions and the like that authenticate the examination data and information of examinees who have taken examinations at health check institutions or medical care institutions and the like, “0003 Examiner information table” to store detailed data and information related to examinees, “0004 User information table” to store detailed data and information related to users who use various highly reliable data registered by the examinee, and so forth stored in association with each other.

In the “health check institution information table”, for example, health check institution IDs that identify “0001 XX General hospital”, “0002 XX University hospital” and so forth are stored in association with each other, to provide various information such as names, summary data, contact information and so forth of the facilities of each institution, related to the health check institution, or the health check institution and the like.

In the “authentication information assignment institution information table”, for example, authentication institution IDs that identify “0001 authentication institution A”, “0002 Authentication institution B” and so forth are provided as detailed data and various information related to each authentication information assignment institution that authenticates health check institutions and medical care institutions and the like are stored in association with each other.

In the “Examinee information table”, for example, “0001 Examinee ID”, “0002 examinee ID” and the like are stored as information for identifying the examinees of the highly reliable data transaction system 1. In the “Examinee management table”, various information related to “0001 Examinee ID” is stored, on a per examinee basis, and, for example, “001 Examinee information”, which serves as examinee-related identification information for identifying examinees (for example, IDs, genetic data, fingerprint data, retina data, vein data, etc.), “001 Body measurement data”, which is linked with “001 Examinee information”, and which serves as measurement data related to the examinee's body such as, for example, the examinee's height, weight, visual acuity, hearing, grip strength and so forth, “002 Health check information,” which serves as information related to medical care such as how regularly the examinee goes to hospitals, “003 Treatment information,” which serves as information related to the treatment that the examinee is receiving or used to receive, “004 Medication information”, which serves as information related to the type, amount, duration and so forth of medications taken by the examinee, “005 Body part information”, which serves as information related to each part of the examinee's body, such as the brain, eyes, teeth, respiratory organs, digestive organs, heart, formation, and so forth, “006 Lifestyle information”, which serves as information related to daily life such as what exercise the examinee does, whether or not the examinee smokes, how much the examiner drinks, gym, whether the examinee does exercise such as running, swimming and so on, and the health record of the examinee, and “007 Admitted body data”, which serves as data the examinee admits as his/her own body data, and as various data entered by the examinee himself/herself, are stored in association with each other.

Furthermore, in the “Examinee information table”, for example, “002 My-page Information”, which shows the reference page on a cloud dedicated to the examinee, “003 Password” for accessing my-page, “004 Health check result/highly reliable data”, which stores health check result information and highly reliable data of the examinee, “005 Conditions of use”, which serves as various conditions (for example, the range of use, the duration of use, the location of use, the charge for use, the points for use, the conditions for exchange, etc.) regarding the use of highly reliable data, Generated highly reliable data of the examinee and “006 highly reliable data” as information on authentication, “007 History of use”, which serves as various histories of services and products related to examinees and used by users, and “008 Update date information”, which serves as the date and time of update, such as update of my-page information and password for logging in to my-page and the like are stored in association with each examinee ID.

In the “user information table”, for example, “U0001 XX Life insurance company”, “U0002 XX Property damage insurance company”, and so forth, which serve as various information such as names related to users, business overviews, representative contact information, maps, and so forth, are stored. For example, as information to be linked with “U0001 XX Life insurance company”, various information such as features, requirements for subscription, prices, etc. regarding the services and products handled are stored as “001 handling services/products”. Furthermore, as various information such as detailed transaction information linked with “001 handling services/products”, user-related authentication information, transaction history-related information, and so forth, for example, “001 Conditions/targets”, “002 Price/points”, “003 Authentication information”, “004 History of use”, and so forth are stored in association with each user ID.

At the transaction management information database 2b, for example, the data manager operates the transaction management device 2a to update various information stored in the transaction management information database 2b. The transaction management information database 2b may store, for example, standardization model information for standardizing the result data of health checks (health check result data) of the examinees (person taking a health check) took in the health check institution (not shown)

(Health Check Institution Database 3c)

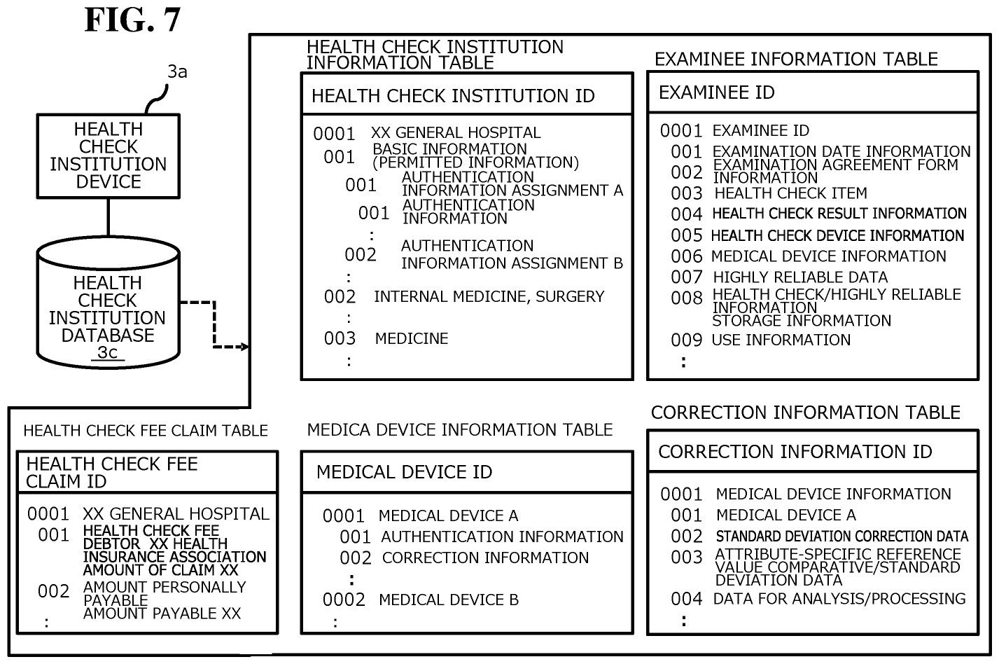

Next. with reference to FIG. 7, an example of the health check institution database 3c connected to the health check institution device 3a according to one embodiment will be described.

As shown in FIG. 7, the health check institution database 3c stores, for example, information related to each health check institution. In the health check institution database 3c, for example, a “Health check institution information table” to identify sections in each health check institution, a “Medical device information table” to identify the medical devices used in medical examinations and the like, an “Examinee information table” to store the consent form for the examinee, health check information to show the test results, and so forth, in association with each other, an “Examinee information table” to store various information related to examinees, a “Correction information table” to store information for various corrections of data measured upon medical examinations, a “health check fee and claim table” to store various information related to health check institutions are stored in association with each other.

In the “health check institution information table”, for example, information related to “0001 XX General hospital” is stored as various information such as basic information (license information) related to health check institution, health check institution, and the like, and information on authentication. In the “Health check institution information table”, for example, “001 Basic information (license information)”,“001 Authentication institution information” linked with “001 Basic information (license information)” and “001 Authentication information” linked with “001 Authentication institution information” and the like are stored. Also, as information related to each section and facility of XX general hospital, “002 Internal medicine/surgery”, “003 Medicine”, and the like, as information related to “0001 XX General hospital” are linked with each other. Furthermore, as information related to various medical devices provided in each department, for example, “0001 Medical device A” and the like are stored in association with the health check institution ID.

Information related to “0001 Medical device A” is stored in the “Medical device information table”. For example, “001 Date of Installation”, “002 Information about person in charge”, “00n Correction information”, which serves as correction data related to the medical device A and the like are stored in association with medical device IDs.

Information related to “0001 Examinee ID” is stored in the “Examinee information table”, and for example, “001 Examination date/time information” as information related to the examination of the examinee, “002 Examination consent form information” as information related to examination and personal permission for the use of highly reliable data, “003 Health check items” and “004 Health check result information” as information related to the health check items and health check results, “005 Health check institution information” and “006 Medical device information” as information for sending health check information and receiving authentication, “007 Highly reliable data”, which is information related to the examinee's highly reliable data generated and authentication, “008 Health check information storage location information” as information related to the location for storing health check information and medical care information of the examinee and the authentication result of the health check information and the like, are stored in association with examinee IDs. Note that the authentication result (highly reliable data) of health check information authenticated by the predetermined authentication information assignment institution may be stored in, for example, the authentication information assignment database 5b, which will be described later.

The “Correction information table” stores information related to “001 medical device information”, provided in each health check institution, and for example, “0001 Medical device A” as the product number of each medical device used in examinations, “002 Health check result information”, as result information such as data, images, videos and the like, before and after correction of health check information, diagnosis results by doctors, “003 Standard deviation correction data” and “004 Analysis processing data”, as standard deviation correction data related to health check information, such as correction of statistical data output from various medical devices and applications used in medical devices, data for analysis processing related to applications for analysis processing, attribute-specific reference value comparative standard deviation data, transition data, analysis data, and so forth are stored in association with correction information IDs.

The “health check fee claim table” stores information related to “0001 XX General hospital” regarding the claims of each health check institution for example, “001 Health check fee debtor, XX health insurance association, claim amount XX”, “002 Amount personally payable, amount payable XX”, and so forth as cases related to the claims of the health check institution are stored in association with medical examination fee claim IDs.

(Authentication Information Assignment Database 5b)

Next, with reference to FIG. 8, an example of the authentication information assignment database 5b connected to the authentication information assignment device 5a according to the embodiment will be described.

As shown in FIG. 8, the authentication information assignment database 5b stores, for example, various information related to the authenticated medical institution and the examinee. In the authentication information assignment database 5b, for example, an “Authentication management table” to store various information related to various health check institutions or health check institution authentication, an “Examinee management table” to store various information of each examinee, a “User time table” to store various information of users who are each a service provider are stored in association with each other.

In the “Authentication management table”, information related to each health check institution that has been requested to authenticate the health check information of the examinee is stored as, for example, “01 Health check/medical examination/medical care”, “02 Nursing”, and the like, as category information to be authenticated. Furthermore, in association with each category information, for example, “0001 XX General hospital” and “0002 XX University hospital” are recorded as identification information related to the health check institution where authentication has been requested or authentication has been performed. Furthermore, as various detailed information linked with “0001 XX General hospital”, and related to health check institutions, for example, “001 Address, telephone number, . . . ”, “002 Representative/administrator”, and so forth are stored. Furthermore, as detailed information related to authentication, for example, “002 Authentication information”, “003 Date of authentication”, “004 Date of expiration”, “005 Subject of authentication”, and so forth are stored in association with each other. Furthermore, in “005 Subject of authentication”, for example, “001 Authenticator”, “002 Authentication device” and the like are stored in association with the subject of authentication.

For example, when the administrator of the authentication information assignment institution operates the authentication information assignment device 5a, the authentication information assignment database 5b updates various information stored in the authentication information assignment database 5b.

(Highly Reliable Data Storage Database 7b)

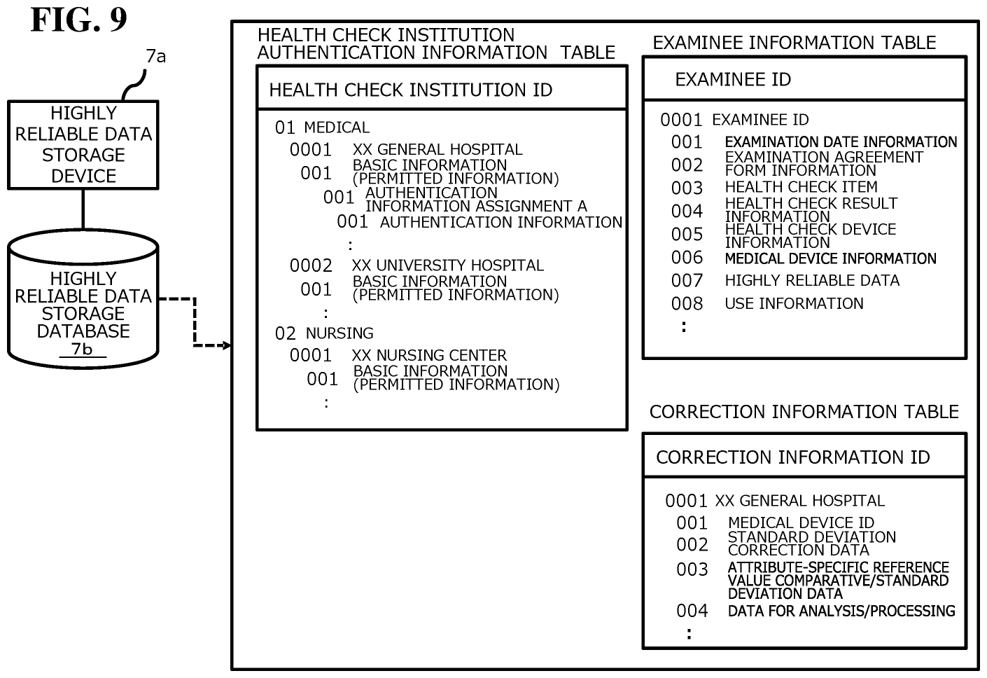

Next, with reference to FIG. 9, an example of the highly reliable data storage database 7b connected to the highly reliable data storage device 7a according to the embodiment will be described.

As shown in FIG. 9, for example, Highly reliable data, and medical examination/examinee consent data from health check institutions (hospitals, etc.), medical data-providing institutions such as testing institutions, (medical examination/medical care data), highly reliable data of health check institutions (hospitals, etc.), medical data-providing institutions such as testing institutions, highly reliable data of examinees, highly reliable data of users, correction data for various highly reliable data and the like are stored in the highly reliable data storage database 7b. Highly reliable data refers to data provided from institutions that are certainly trusted.

In the highly reliable data storage database 7b, for example, “Health check institution information table” to identify a plurality of health check institutions and medical care institutions, “Medical device information table” to identify the medical devices used in each institution for medical examinations and medical care, “Examinee information table”, in which the consent form for the examinee and the medical care staff in each institution, health check information to show the test result and the like are stored in association with “0001 Examinee ID”, and “Correction information table” to store information for various corrections of data measured upon examinations are stored in association with each other.

In the “health check institution information table”, for example, as a plurality of health check institutions, information related to “0001 XX General hospital” is stored as basic information (license information) related to various health check institutions, or health check institutions, medication institutions, nursing care institutions, and the like, authentication information, and the like is stored. In the “health check institution information table”, for example, “001 basic information (license information)”, and the like are stored in association with “001 Authentication institution information” linked with “001 Basic information (license information)” “001 authentication information” linked with “001 Authentication institution information” and the like are stored.

Information related to “0001 Examinee ID” is stored in the “Examinee information table”, and for example, “001 Examination date/time information” as information related to the examination of the examinee, “002 Examination consent form information” as information related to examination and consent to use your highly reliable data, etc. “003 Health check item” and “004 Health check result information”, as information related to the health check items and health check results, “005 Health check institution information” as information for sending health check information and receiving authentication, “006 Medical device information”, and “007 Highly reliable data” as information related to the highly reliable data of the examinee generated and authentication, “008 Health check information storage location information” as information related to the location for storing health check information and medical care information of the examinee and the authentication result of medical information and the like are stored in association with examinee IDs.

The “Examinee information table” may include, for example, genetic data to show genetic information of the examinee tested at the health check institution, the authentication information assignment institution, or elsewhere (not shown). Note that the authentication result (highly reliable data) of the health check information authenticated by the predetermined authentication information assignment institution may be stored in, for example, the authentication information assignment database 5b, which will be described later.

Information related to “001 medical device information” is stored in the “Correction information table”, and for example, “0001 Medical device A” as the product number of each medical device used in examinations, “002 Health check result information” as result information such as data, images, videos and the like, before and after correction of health check information, diagnosis results by doctors and the like, “003 Standard deviation correction data” and “004 Analysis processing data”, as standard deviation correction data related to health check information, such as correction of statistical data output from various medical devices and applications used in medical devices, data for analysis processing related to applications for analysis processing, transition data, analysis data, and so forth are stored in association with correction information IDs.

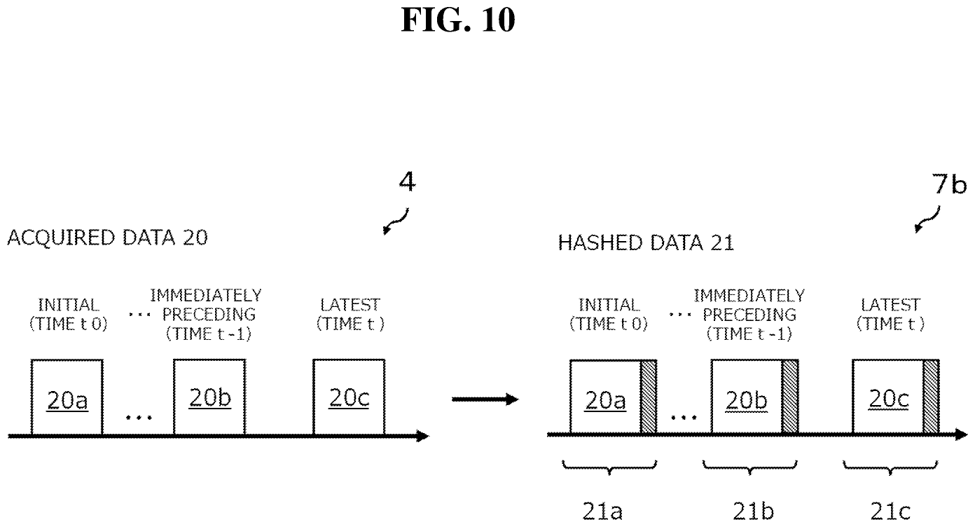

Here, with reference to FIG. 10, as the highly reliable data of the present invention according to one embodiment or various types of acquired data, for example, an example of hashing of acquired data 20 will be described.

The examinee terminal 4 according to one embodiment acquires, for example, acquired data 20c that is sequentially generated at latest time t, acquires acquired data 20b that is sequentially generated at time t-1 immediately before time t, and receives a medical examination. It is assumed that the acquired data 20a is acquired at the initial time t0 of the personal terminal 4.

The examinee terminal 4 acquires, for example, data related to the operation status or the operation result of the examinee terminal 4 which is sequentially acquired in the examinee terminal 4. The data related to the operating status is, for example, device data acquired by a sensor provided in the examinee terminal 4, environmental data, or various measured data, as well as data exchanged by, for example, GPS, temperature, temperature, or serial communication. The data related to the operation status or the operation result acquired in the examinee terminal 4 may be, for example, data of results measured or instrumented by the examinee terminal 4, Furthermore, individuals are subject to examinations, the data may be acquired per examinee.

The acquired data 20a to 20c acquired in the examinee terminal 4 may be, for example, data that is continuously generated in time series, highly reliable data that is continuously generated, or various acquired data. In addition, the acquired data 20a to 20c may be various data or information operated and licensed by the examinee terminal 4, including, for example, sensing data acquired in the examinee terminal 4, operation data, environmental data (temperature, humidity, etc.), logs, history of use and suchlike data.

For example, when new acquired data 20c is acquired at latest time t, the examinee terminal 4 calculates hash data from the acquired data 20b acquired at time t-1, immediately before the newly acquired data 20c, generates new hash data at time t, based on the calculated hash data of the acquired data 20b and the newly acquired data 20c. For example, upon generating new hash data, the examinee terminal 4 attaches the newly generated hash data to the acquired data 20c newly acquired in time t, and generates hashed data 21 of the acquired data 20c (hashed highly reliable data).

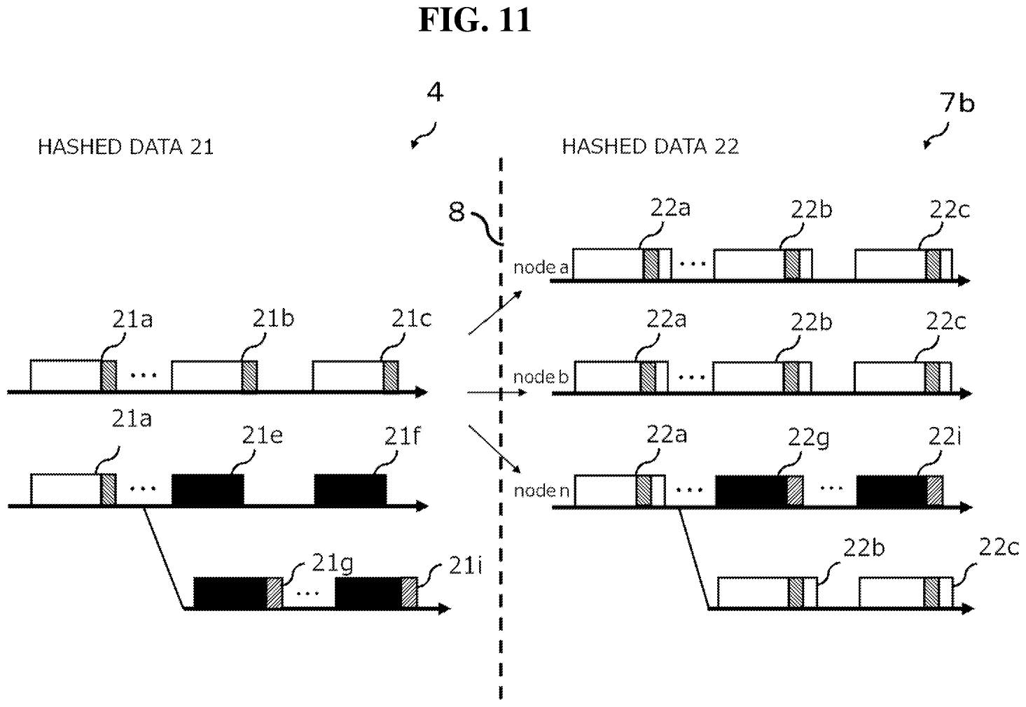

Next, with reference to FIG. 11, an example of data management in the blockchain in the transaction management system 2 or the highly reliable data storage system 7 according to one embodiment of the present invention will be described. The examinee terminal 4 generates hashed data 21c, in which new hash data is added to, for example, the newly acquired data 20c. The examinee terminal 4 performs the process for outputting the hashed data 21c, to which new hash data is added, to the transaction management system 2 or the highly reliable data storage system 7, and outputs the newly acquired data 20c to the transaction management system 2 or the highly reliable data storage system 7, as hashed data 21.

Between the transaction management system 2 or the highly reliable data storage system 7 and the examinee terminal 4, for example, as described above, by means of known server management or the like, data communicated on each node is centrally managed by an effective security method in the transaction management system 2 or in the highly reliable data storage system 7, by means of SSH30 or the like, for example.

Here, for example, if the hashed data 21 output from the examinee terminal 4 is normal, hashed data 21a to hashed data 21c are recorded in the distributed network 8, and stored in in the transaction management system 2 or in highly reliable data system 7 in a distributed manner. If the hashed data 21 output from the examinee terminal 4 has been tampered with, for example, based on the hash data of the acquired data 20b acquired immediately before and the latest acquired data 20c, no new hash data corresponding to the latest acquired data 20c will be generated. Consequently, hashed data 21a, tampered data 21e and tampered data 21f are generated.

Also, for example, even if tampered hash data is generated from the tampered data 21e, these are not linked with the hashed data 21a immediately before normal. Consequently, it is deleted as 21g of tampered data.

By this means, in the transaction management system 2 or the highly reliable data storage system 7, for example, if the hashed data 21 has been generated successfully based on existing blockchain technology, each hash data of the hashed data 21a to the hashed data 21c that are sequentially output from the examinee terminal 4 is a blockchain corresponding to the acquired data 20 acquired in the examinee terminal 4.

The transaction management system 2 or the highly reliable data storage system 7 judges that the data is normal, based on existing blockchain technology, and sequentially records the data as blockchain-connected blocks. For example, when outputting to the transaction management system 2 or the highly reliable data storage system 7, the examinee terminal 4 may output the hashed data 21 to the transaction management system 2 or the highly reliable data storage system 7 only when newly generated hash data is added to the hashed data 21.

The examinee terminal 4 identifies, for example, the hash data added to the hashed data 23, and separate this hash data from the hash data of the immediately preceding acquired data 20a. Given the hashed data 21a to 21c, the examinee terminal 4 may output, for example, the hashed data 21a to 21c to the transaction management system 2 or the highly reliable data storage system 7.

The examinee terminal 4 refers to, for example, the data following the hashed data 21a, and, if the data is identified as being, for example, non-hashed data 21e or 21f, or tampered data 21g to 21i, to which tampered hash is added, the examinee terminal 4 may, for example, manage various highly reliable data, so as not to output these tampered data 21g to 21i to the transaction management system 2 or the highly reliable data storage system 7.

The transaction management system 2 or the highly reliable data storage system 7, for example, consults the hashed data 21 output from the examinee terminal 4, and judges whether or not new hash data is added. The transaction management system 2 or the highly reliable data storage system 7 records the hashed data 21 in, for example, the blocks connected by the blockchains built in the distributed network 8, based on the result of judgement.

In the transaction management system 2 or the highly reliable data storage system 7, for example, when hashed data 21 output from the examinee terminal 4 is continuous with a node a (when hash data newly generated in the examinee terminal 4 is included), the hashed data 21a to 21c are recorded in the blockchains. The transaction management system 2 or the highly reliable data storage system 7, for example, hashes and records each of the hashed data 21a to 21c, output from the examinee terminal 4, in the blocks connected by the blockchains, based on existing blockchain technology.

The transaction management system 2 or the highly reliable data storage system 7 judges whether the hash data added to the hashed data 21 output from the examinee terminal 4 is hash data calculated from acquired data 20b that was acquired immediately before the newly acquired data 20c, and, furthermore, is new hash data generated based on the hash data calculated, and the newly acquired data 20c, with reference to each data. Note that the hash data to be generated first in the examinee terminal 4 may be, for example, hash data that is generated from the acquired data 20a that is acquired first and the initial data that is set in the examinee terminal 4 in advance.

The transaction management system 2 or the highly reliable data storage system 7 judges whether the hash data generated in the examinee terminal 4 is added to each hashed data 21, by referring to, for example, each hash data of the latest hashed data 21c and the immediately-preceding hashed data 21b, or by referring to each data or numerical value and matching and comparing these data against each other. The transaction management system 2 or the highly reliable data storage system 7, after receiving the hashed data 21b at the node a, for example, generates, for example, hashed data 21b and nonce, or new hashed data 21c, as a block of the blockchains.

The transaction management system 2 or the highly reliable data storage system 7 may generate reference indices and manage the data by, for example, storing the pairs of hashes and nonces, generated in the node a, after the output from the examinee terminal 4, as blocks connected by blockchains, in respective databases of the highly reliable data storage system 7.

The transaction management system 2 or the highly reliable data storage system 7 consults each data in each block comprised of, for example, node b and node n, and matches and compares these data (for example, hashed data 21b and tampered data 12g) against each other. The transaction management system 2 or the highly reliable data storage system 7 consults each data in each block constituting node b and node n, for example, and, based on the result of comparison for seeing whether these data match, identifies between the hashed data 21a and the tampered data 21g, for example.

The transaction management system 2 or the highly reliable data storage system 7 consults each data of, for example, hashed data 21a and tampered data 21g, based on existing blockchain technology, and, when judging that each nonce and hash data do not match, removes the tampered data 21g that does not match, and performs the process of making the hashed data 21b continuous as a block, as normal hashed data 21 to follow the hashed data 21a.

The transaction management system 2 or the highly reliable data storage system 7 repeats this process, so that, even when the data following hashed data 21a of the node n is tampered data 21g, it is possible to use the hashed data 21 output from the examinee terminal 4 as normal received data and processing the data as blocks connected by the blockchains.

Also, the transaction management system 2 or the highly reliable data storage system 7 may acquire in advance, for example, the hash of the acquired data 20b of time t, which is immediately before time t+1 of the hashed data 23 of the latest acquired data 20c of time t+1, generated in the examinee terminal 4. In this case, for example, the transaction management system 2 or the highly reliable data storage system 7 places the immediately preceding acquired data 20b in a secret state, and manages the hash data, keeping this secrete state.

By this means, even when the output of hashed data 21 from the examinee terminal 4 to the transaction management system 2 or the highly reliable data storage system 7 ceases, the transaction management system 2 or the highly reliable data storage system 7 is still able to acquire newly acquired data in the examinee terminal 4, based on the hash data of the immediately preceding acquired data 20b that is acquired in advance.

By this means, even when the output of hashed data 21 from the examinee terminal 4 ceases, the transaction management system 2 or the highly reliable data storage system 7 is still able to identify the tampering between hashed data 21, based on the hash data of the immediately preceding acquired data 20b that is acquired in advance, and record the hashed data 21 in the blocks connected by the blockchain built in the distributed network 8.

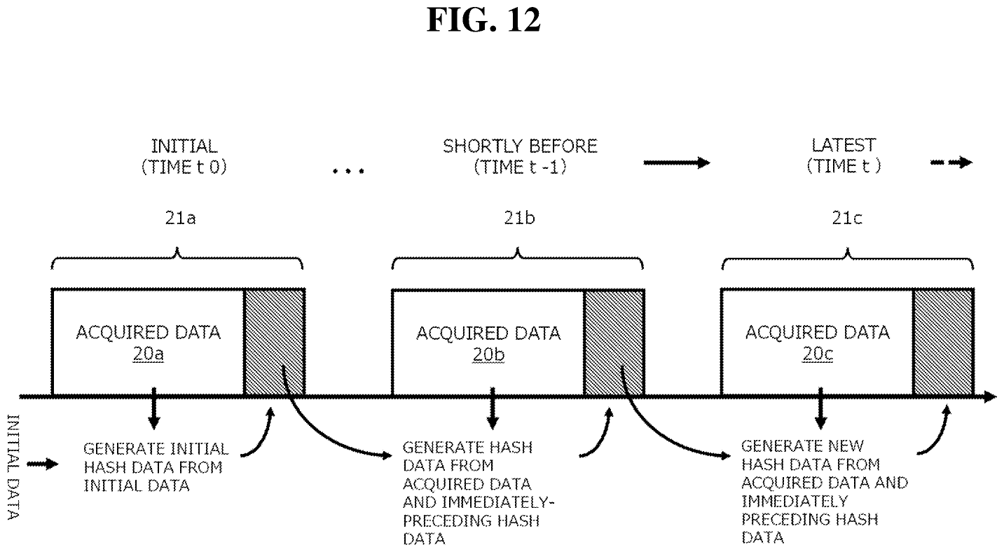

Here, with reference to FIG. 12, an example of the operation of adding hash data in the examinee terminal 4 according to one embodiment will be described. For example, when the examinee terminal 4 acquires data at the initial stage (time t0), the examinee terminal 4 generates initial hash data (that is, for example, managed by the transaction management system 2 or the management database 3 of the highly reliable data storage system 7), based on the initial data assigned to the examinee terminal 4 in advance, and the acquired data 20a first acquired in the examinee terminal 4.

The examinee terminal 4 adds the hash data of the initial data, generated based on initial data and the acquired data 20a, to the head of the acquired data 20a, and generates initial hashed data 21a. The examinee terminal 4 outputs this generated hashed data 21a to the transaction management system 2 or the highly reliable data storage system 7. After that, when the examinee terminal 4 acquires, for example, the latest data as acquired data 20c at time t, the examinee terminal 4 generates new hash data to add to the acquired data 20c, based on the hash data that is immediately preceding (immediately preceding hash data) and that is added to the immediately-preceding hashed data 21b (time t-1), and the newly acquired data 20c.

The examinee terminal 4 adds the generated new hash data to the acquired data 20c. By this means, the examinee terminal 4 is associated with the acquired data 20 that is sequentially acquired in the examinee terminal 4. In the examinee terminal 4, for example, when acquired data 20 is replaced with tampered data in the middle of acquiring the acquired data 20, the value of the original acquired data for generating the hash data will be different. Since different hash data is generated in the examinee terminal 4, by comparing these data, the examinee terminal 4, the transaction management system 2, or the highly reliable data storage system 7 can identify tampered or tampered hash data.

For example, the examinee terminal 4 may output the hashed data 21 generated in the examinee terminal 4 to the transaction management system 2 or the highly reliable data storage system 7, at each timing hashed data 21 is generated. The examinee terminal 4 may, for example, output a plurality of hashed data 21 generated, at regular time intervals, to the transaction management system 2 or the highly reliable data storage system 7.

When the examinee terminal 4 outputs hashed data 21 to the transaction management system 2 or the highly reliable data storage system 7, at what timing the hashed data 21 to be output to the transaction management system 2 or the highly reliable data storage system 7 is generated is unspecified.

Whatever output is provided from the examinee terminal 4, new hash data to add to the acquired data 20c, newly acquired in the examinee terminal 4, is generated based on the immediately-preceding hash data of the acquired data 20b, added to the acquired data 20b acquired immediately before that, and the acquired data 20c that is newly acquired. After that, the examinee terminal 4 adds the generated new hash data to the head of the acquired data 20c.

The examinee terminal 4 transmits the hashed data 21c, to which the new hash data is added, to the transaction management system 2 or the highly reliable data storage system 7. In this way, every time new acquired data 20 (for example, acquired data 20c) is acquired in the examinee terminal 4, the examinee terminal 4 repeats the process of consulting the immediately-preceding hash data added to the acquired data 20 (for example, acquired data 20b) acquired immediately before, and the newly acquired data 20c, regularly generating new hash data based on the value of each data, and adding the generated new hash data to the head of the acquired data 20c.

The examinee terminal 4 calculates hash data from highly reliable data, following the above process, and generates new hash data from the hash data of the immediately preceding highly reliable data and the newly acquired highly reliable data. The examinee terminal 4 adds the new hash data to the newly generated highly reliable data, and records the hashed highly reliable data in blocks connected by the blockchains built in the distributed network 8.

(Reference Database)

A training model is stored in the reference database stored in the storage unit 103. The training model provides associations between a plurality of data included in past highly reliable data, acquired in advance, and reference information linked with the plurality of data included in the past highly reliable data. For example, past highly reliable data and reference information may be stored in the reference database. The training model is built on machine learning using a plurality of training data, using, for example, past highly reliable data and reference information as a set of training data. As for the learning method, deep learning such as a convolutional neural network is used.

In this case, for example, associations indicate the degree of connection between many-to-many information. The associations are updated as appropriate during the machine learning process. Consequently, the training model shows, for example, a classifier tree with associations (for example, functions) that are optimized based on health check information associated with past highly reliable data and reference information. Consequently, evaluation results for the highly reliable data are generated using the training model built based on all results of evaluating the highly reliable data to be evaluated in the past. By this means, even when the highly reliable data to be evaluated shows complicated medical care conditions and types, it is still possible to generate optimal evaluation results. Also, when the highly reliable data included in the evaluation target is the same as or similar to past highly reliable data, or when it is dissimilar, it is still possible to generate optimal evaluation results quantitatively. Note that, by improving the capability for generalization when implementing machine learning, it is still possible to improve the accuracy of correction of the evaluation data of highly reliable data.

Note that it may be possible, for example, to provide a plurality of associations to show the degree of connection between a plurality of target data included in each past highly reliable data, and each reference information. It is possible to provide associations that correspond to weight variables, for example, when the training model is built on a neural network. The highly reliable data includes, for example, authenticator information related to authenticators to provide authentication, authentication information, including date/time information to show the date and time of authentication, consent information of the examinees, health check institution information for identifying the health check institution where the examinee has been examined, medical devices for acquiring health check information, assignment information related to the correction of standardization, use information related to the use of highly reliable data, reference value comparative standard deviation data, which is provided per examinee attribute, transition data of examinees, or future predicting data of examinees, and/or the like.

A plurality of evaluation data included in past highly reliable data show the same kind of information as the above-mentioned evaluation data. The evaluation data included in past highly reliable data includes, for example, a plurality of evaluation data acquired when the highly reliable data was evaluated in the past.

The reference information is linked to the evaluation data included in past highly reliable data, and shows information related to the medical condition of the evaluation data of the highly reliable data to be evaluated for correction. The reference information indicates, for example, the type and state of a plurality of pieces of information included in the highly reliable data to be evaluated. Note that the specific contents and settings included in the reference information, and the default values of various optimal values can be added or set arbitrarily.

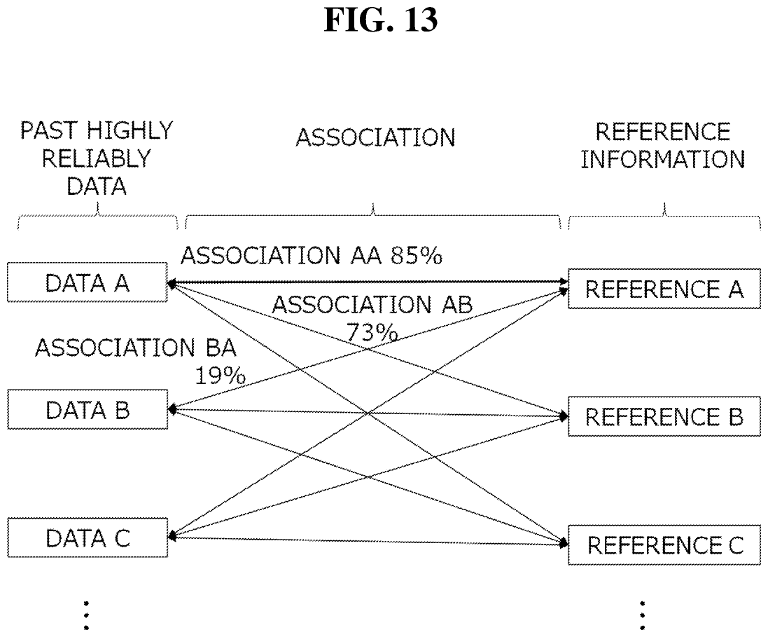

The associations may indicate the degree of connection between the evaluation data included in past highly reliable data, and the reference information, as shown in FIG. 13, for example. In this case, by using associations, it is possible to store the degree of relationship of each of a plurality of data (in FIG. 13, “reference A” to “reference C”) included in the reference information, with each of a plurality of data (“data A” to “data C” in FIG. 13) included in past highly reliable data. Consequently, for example, through associations, it is possible to link a plurality of data included in the reference information with one data included in the evaluation data included in the past highly reliable data, and generate multi-faceted evaluation results.

The association has, for example, a plurality of associations that associate a plurality of data included in the past highly reliable data with a plurality of data included in the reference information. Associations are shown in three or more steps, such as percentage, 10 steps, or 5 steps, and are shown, for example, by line characteristics (for example, thickness, etc.). For example, “data A” included in the evaluation data of the past highly reliable data indicates an association AA “85%” with “reference A” included in the reference information, and “reference B” included in the reference information. The association AB “73%” between and is shown. Also, for example, “data B” included in the past highly reliable data indicates an association BA “19%” with “reference A” included in the reference information. That is, the “association” indicates the degree of connection between each data. For example, the higher the association, the stronger the connection of each data. Note that, when constructing a learning model by the above-mentioned machine learning, the association may be set to have three or more stages of association.

The past highly reliable data may be stored in a reference database by dividing, for example, the past highly reliable data and a plurality of information about the past highly reliable data (not shown). In this case, the association is calculated based on the relationship between the combination of the past highly reliable data and the past highly reliable data and the reference information. Note that, the evaluation data contained in the past highly reliable data is, for example, divided into at least one of the past highly reliable data and a plurality of evaluation data contained in the past highly reliable data in the reference database in addition to the above. It may be remembered.

(Example of Operation of Highly Reliable Data Transaction System 1)

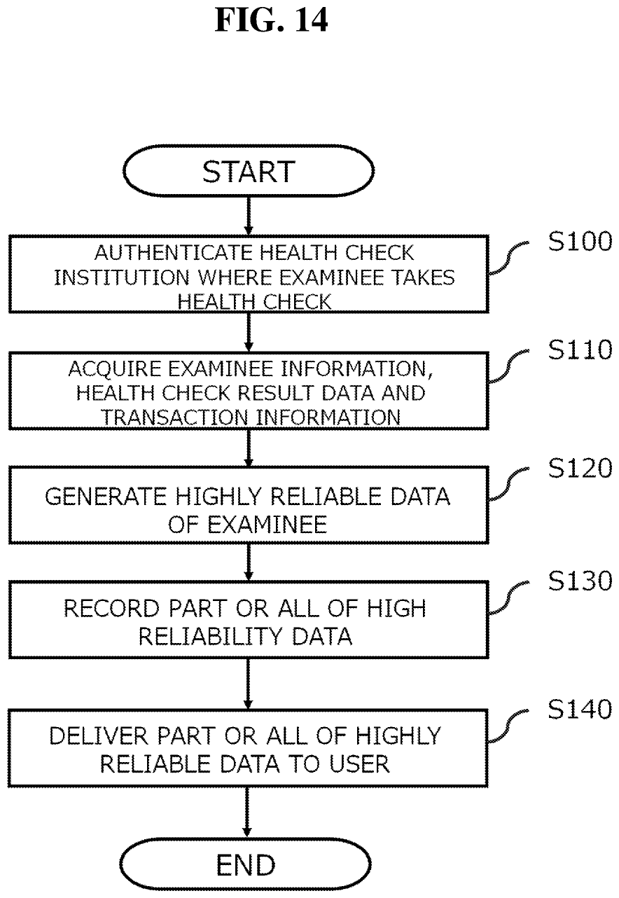

Next, an example of the operation of the highly reliable data transaction system 1 according to the embodiment will be described. FIG. 14 is a flowchart showing an example of the operation of acquiring the authentication information of the highly reliable data transaction system 1 according to one embodiment.

As shown in FIG. 14, the highly reliable data transaction system 1 is a highly reliable data transaction system 1 with a distributed network 8, and includes an authentication means (authentication unit 301) for authenticating that the health check institution that acquires the health check information using the medical device 3b in the health check institution device 3a of the health check institution system 3 is an authenticated health check institution, a first acquisition means (first acquisition unit 302) for acquiring the health check information of the examinee, a highly reliable data generation means (highly reliable data generation unit 303) for generating highly reliable data of the examinee based on the authentication information authenticated in the authentication means and the health check information, a first recording means (first recording unit 304) for recording the highly reliable data generated in the highly reliable data generation means in the blocks connected by the blockchains built in the distributed network 8 and the first recording means (first recording unit 304), and a first delivery means (first distribution unit 305) for delivering part or all of highly reliable data to the users.

<Authentication Means S100>

As shown in FIG. 14, the authentication unit 301 authenticates the health check institution for acquiring the health check information (authentication means S100). The authentication unit 301 receives information related to the health check institution to be authenticated from each health check institution, via the distributed network 8, and authenticates the health check institution based on the received information about the health check institution. The authentication unit 301 consults the authentication information assignment database 5b and judges whether or not it is an authenticated health check institution and whether or not it is an authorized/registered health check institution.

When the authentication unit 301 judges that a health check institution is an authenticated one, the authentication unit 301 issues authentication information for the health check institution, and transmits the issued authentication information to the health check institution system 3. Also, for the medical devices, examinees, or users, too, whether each one is an authenticated device or an authenticated subject is judged, and, if each one is judged as being an authenticated one, authentication information may be issued and transmitted to the health check institution system 3.

The authentication unit 301 may execute an authentication means S110 using, for example, an authentication algorithm, an authentication program or the like stored in the storage unit 103 (not shown). Then, the result of authentication by the authentication means S110 is stored as, for example, “002 Authentication information” in the “authentication management table” of the authentication information assignment database 5b.

The health check institution system 3 receives the authentication information transmitted from the authentication information assignment system 5, and stores the received authentication information as “0001 XX General hospital, 001 Authentication information,” in the “Health check institution information table” of the health check institution database 3c. After the health check information of the examinee is acquired, for example, the health check institution device 3a may added this to the health check information, in order to prove that the examinee has been examined in an authenticated health check institution.

<First Acquisition Means S110>

Next, the first acquisition unit 302 acquires health check information related to the medical care which the examinee took (first acquisition means S110). The first acquisition unit 302 performs a health check, a medical examination and the like, on the examinee, via the health check institution device 3a and the medical device 3b, and, as a result of this, acquires health check information, including at least examination data, medical care data, medication data, or genetic data, related to the examinees.

The first acquisition unit 302 may acquire the result data (health check result data) of the health checks the examinees have taken, from a plurality of health check institutions, via the health check institution system 3.

The health check information, which is the results of the examinees' health checks, is stored in the health check institution database 3c, via the health check institution device 3a, in association with examinee IDs. The first acquisition unit 302 consults various information related to the examinees who have been examined, based on the examinee IDs input via the health check institution device 3a, for example, and acquires various information, linked with the health check information showing the examination results of the examinees, as shown in the examinee information table of the health check institution database 3c.

The first acquisition unit 302 associates part or all of the acquired information as examinee IDs, for each examinee who has been examined. Note that, when the same examinee receives medical examinations for a plurality of items, for example, separate health check information may be acquired in association with the examinee's ID. When health check information is already stored in the health check institution database 3c, the first acquisition unit 302 may acquire the target health check information, from the health check institution database 3c, based on the conditions of use of highly reliable data, or the content of the user's request for use.

<Highly Reliable Data Generation Means S120>

Next, the highly reliable data generation unit 303 generates highly reliable data of the examinees, based on the authentication information and health check information received in the highly reliable data generation means S120 (highly reliable data generation means S120).

The highly reliable data generation unit 303 includes, in the highly reliable data, at least, for example, authenticator information related to the authenticator that performed authentication in the certification unit 301, authentication information, including date/time information to show the date and time of authentication, consent information of the examinees, health check institution information for identifying the health check institutions where the examinees took a health check, medical devices for acquiring health check result data, additional information related to the correction of standardization, use information related to the use of highly reliable data, attribute-specific reference value comparative standard deviation data, provided per examinee attribute, aging data of the examinees, storage location information for identifying the location where the health check result data is stored separately, my-number information for identifying the examinees, health insurance card information of the examinees, or future predicting data of the examinees.

Also, the highly reliable data generation unit 303 includes, as assignment information related to the health check information, for example, standard deviation correction data for the health check result data, attribute-specific reference value comparative standard deviation data, transition data, analysis data, device information or common language that serves as a standard for standardization, or standard model data. Note that, when an examinee has insurance for taking examinations, for example, questions and answers to them, provided from the insurance company in advance, items to confirm with licensed persons, interview result information and so forth may be acquired and included as additional information.

If the information related to and required for the use of highly reliable data is needed in the health check institution device 3a or to review the insurance or the like, the highly reliable data generation unit 303 may receive as input that information from each user terminal 6. The highly reliable data generation unit 303 may include information such as, for example, the review items, as well as answers to the review items, the signatures of the examinees and so forth, and store these in the area that is identified as “001 Examinee ID” in the “Examinee management table” of the authentication information attached database 5b.

Also, the highly reliable data generation unit 303 generates corrected highly reliable data related to the examinees, based on the authentication information authenticated in the authentication unit 301 and the corrected health check information of the examinees.

Here, an example of the operation of correcting highly reliable data in the highly reliable data generation unit 303 in the highly reliable data transaction system 1 will be described.

If there is a correction (or a change, deletion, etc.) of the highly reliable data of an examinee who has been treated in a health check institution authenticated by the authentication unit 301, the highly reliable data generation unit 303, based on the corrected health check information of the examinee, acquires the highly reliable data of the blockchains built in the distributed network 8, recorded by the first recording unit 304, changes the numerical values and information of the items to be corrected, and generates corrected highly reliable data.

The corrected highly reliable data corrected in the highly reliable data generation unit 303 is recorded by the first recording unit 304 in the blocks connected by the blockchains built in the distributed network 8, together with the highly reliable data before the correction. Consequently, the reliability of the highly reliable data and corrected highly reliable data to be recorded on the blockchains can be ensured based on the examinee's will, and the working time required for analyzing and processing the data to use can be shortened. By this means, it is possible to develop services and products that are suitable for customers.

<First Recording Means S130>

Next, the first recording unit 304 records the highly reliable data generated in the highly reliable data generation means S120, in the blocks connected by the blockchains built in the distributed network 8 (first recording means S130). Also, in the first recording unit 304 records information related to the examinee's highly reliable data and that indicates, for example, that doctor A working at the health check institution system 3 has filled in the examinee's electronic medical record input via the health check institution device 3a, that doctor B has referenced the electronic medical record, that the examinee has granted health check institution B the authority for viewing the health check information and highly reliable data that show the results of his/her own health check, in the blocks connected by the blockchains.

<First Delivery Means S140>

When transaction information related to the acquisition of part or all of the highly reliable data recorded by the first recording means S130 is acquired, the first delivery means S140 distribute part or all of the highly reliable data to the user, depending on the transaction information (first delivery step 140).

The first delivery means S140 may deliver, for example, highly reliable data or corrected highly reliable data and/or the like, given by someone besides the examinee, such as, for example, a representative delegated, entrusted or requested by the examinee. Also, the first delivery means S140 may deliver the entire file of the highly reliable data requested for use, or deliver only part or the corresponding part of the highly reliable data. The first delivery means S140 acquires the target highly reliable data and corrected highly reliable data, recorded in the highly reliable data storage database 7b or the blockchain, and delivers them to the user.

By this means, for example, the authentication unit 301 authenticates the health check institution to acquire health check information, the first acquisition unit 302 acquires the health check information, the highly reliable data generation unit 303 generates the highly reliable data of the examinee based on authentication information and the health check information, the first recording unit 304 records the highly reliable data of the examinee in the blocks connected by the blockchains built in the distributed network 8, and the first delivery unit 305 delivers part or all of highly reliable data to the user, and thereupon the series of operations in the transaction management system 2, the health check institution system 3, the authentication information assignment system 5, and the highly reliable data storage system 7 of the highly reliable data transaction system 1 according to one embodiment is finished.

(Example of Operation of Highly Reliable Data Transaction System 1)

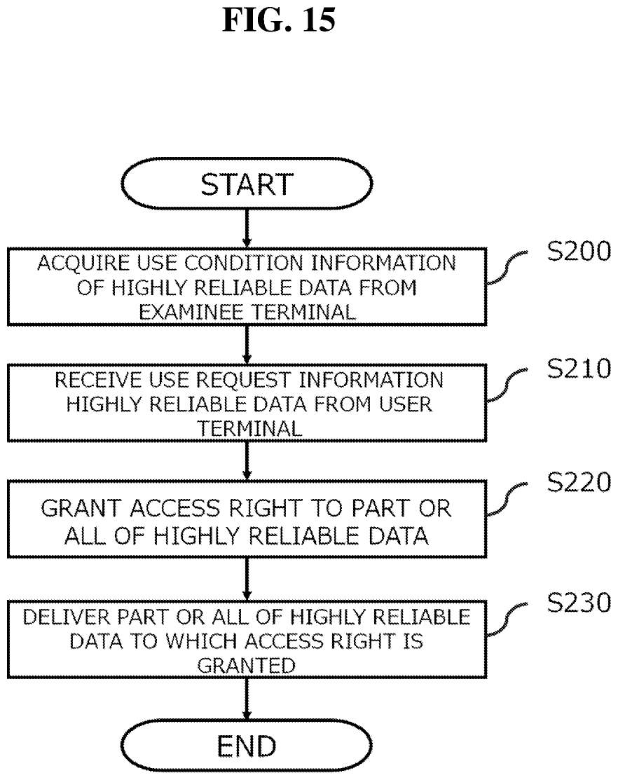

Next, an example of the operation of the highly reliable data transaction system 1 according to the embodiment will be described. FIG. 15 is a flowchart to show an example of the operation for granting access rights in the highly reliable data transaction system 1 according to one embodiment.

The highly reliable data transaction system 1 further has a second acquisition means (second acquisition unit 201) for acquiring the use condition information related to the conditions of use of highly reliable data from the examinee terminal of the examinee, a receiving means (receiving unit 202) for receiving use request information that collects requests from user terminals that wish to use highly reliable data, an access right granting means (access right granting unit 203) for granting access rights to the highly reliable data recorded in the recording means based on the acquired use condition information and the received use request information, and a delivery means (first delivery unit 305) for acquiring the highly reliable data, to which access rights are granted from the access right granting means, from the blocks connected by the blockchains built in the distributed network recorded by the first means, and delivering the acquired highly reliable data to the user terminal.

<Second Acquisition Means S200>

The second acquisition means S200 acquires the use condition information related to the conditions of use of highly reliable data by the second acquisition unit 201, via the examinee terminal 4 of the examinee (second acquisition step 200). The second acquisition unit 201 consults the “Examinee information table”, stored in the transaction management information database 2b, based on the examinee ID and password from the examinee terminal 4, and acquires the examinee's “002 My-page information”. Based on the Web screen information (not shown) stored in this “002 My-page information”, the examinee terminal 4 inputs and outputs various information. The second acquisition unit 201 acquires information related to the use of the examinee's highly reliable data, input from the examinee via the examinee terminal 4, via my-page, provided on the Web prepared per examinee. Note that, for confirmation of the examinee, for example, authentication may be performed based on the examinee's my-number.

In the second acquisition means S200 may acquire, for example, various other information from the examinees through each examinee's my-page. Other information includes, for example, various information related to examinees, stored in the “Examinee information table” stored in the authentication information assignment database 5b.

Also, the second acquisition unit 201 acquires, consent information related to the transactions of the examinee's highly reliable data (for example, whether or not standardization is possible, applicability to transactions, etc.), via the examinee terminal 4 of the examinee.

Also, the second acquisition unit 201 acquires the estimation information related to the medical care of the examinee from the health check institution. The estimation information acquired in the second acquisition unit 201 is recorded, via the examinee terminal 4, based on this estimation information acquired in the second acquisition unit 201 and the highly reliable data of the examinee recorded by the first recording unit 304, in the blocks connected by the blockchains built in the distributed network 8, as support content related to the examinee's treatment.

The support content recorded by the first storage unit 304 may be disclosed to the cloud founder, cloud lender, or financial institution, through a site on the Web provided by the transaction management system 2, via the examinee terminal 4 of the examinee, and receives support.

Here, an example of standardization of highly reliable data and transaction operation in the highly reliable data transaction system 1 will be described.

The second acquisition unit 201 acquires the consent information related to the transactions of the examinee's highly reliable data (for example, whether or not standardization is possible, applicability to transactions, etc.), via the examinee terminal 4 of the examinee, and then the highly reliable data generation unit 303 acquires the highly reliable data generated in the highly reliable data generation unit 303, from the blocks connected by the blockchains built in the distributed network 8, recorded by the first recording unit 304, based on the consent information acquired in the second acquisition unit 201.

Next, the standardization process unit 303a standardizes the highly reliable data acquired in the second acquisition unit 201. The standardization process unit 303a standardizes a plurality of health check items of the health check information based on the consent information of the examinee, acquired in the second acquisition unit 201 (other health check items, testing items, etc.). In the standardization process of highly reliable data in the standardization process unit 303a, for example, the standardization process is performed using information such as each item, numerical value, parameter, provider and so forth, set in advance or selected by the examinee.

The standardization process may be carried out so that information such as numerical values, parameters, providers, and so forth authenticated by and input from the transaction management device 2a, health check institution device 3a, authentication institution device 5a, highly reliable data storage device 7a, and other authentication institutions.

Next, in the transaction unit, the highly reliable data of the person taking a health check standardized in the standardization process unit 303a is placed in transaction, for the use and utilization of the standardized highly reliable data, based on the use condition information acquired in the second acquisition unit 201. Various transactions in the transaction unit will be described later.

<Receiving Means S210>

The receiving means S210 receives a use request for highly reliable data, as use request information, from the user terminal 6 requesting use of the highly reliable data of the examinee, in the receiving unit 202 (second receiving step 210). For example, the user terminal 6 pre-registers the use of highly reliable data of the examinee, through the user terminal 6, by a general user registration method such as Web registration or mail registration, and the result is stored in the “user information table” of the transaction management information database 2b of the transaction management system 2. In the use request information, for example, information such as the type or kind, the number of people, the period and so forth may be specified, in addition to, for example, information related to the examinee of the highly reliable data desired for use, and the receiving unit 202 receives the information the user wants, via the user terminal 6.

The use request information that is received as input from the user terminal 6 is recorded in the blocks connected by the blockchains built in the distributed network 8. Examines of the blockchains built in the distributed network 8 can consult the use request information that their own examinee terminals 4 indicate.

<Access Right Granting Means S220>

Access rights to the highly reliable data recorded by the first recording unit 304 are granted based on the use condition information acquired in the received means S210, and the received use request information (access right granting step 220). The access right granting unit 203, for example, grants access rights to each highly reliable data or part of the highly reliable data, on a per user terminal ID basis, based on the condition contents set in “005 Conditions of use”, stored in the “Examinee management table” of the transaction management information database 2b. The access rights granted per user terminal may be stored in the area set in the “006 Authentication information” recorded in the “Examinee management table”, for example.

By this means, the second recording unit 204 enables the use of highly reliable data of the examinee from the user terminal 6. The status of use permission by the examinee terminal 4 and the use of highly reliable data of the examinee by the user terminal 6 are recorded in the “007 History of use”, stored in the “Examinee management table”. The examinee can change the access rights to his/her highly reliable data based on the “002 My-page information”, identified by the examinee ID, through the examinee terminal 4.

<Second Delivery Means S230>

The second delivery means S230 transacts part or all of the highly reliable data, to which access rights were granted by the access right granting means S220, based on the use condition information, the use request information and the access rights, and delivers part or all of the highly reliable data to the user terminal 6, depending on the result of the transaction (second delivery step 230). The delivery means S230 may deliver highly reliable data, corrected highly reliable data and the like, based on the access rights granted by the someone apart from the examinee, such as, for example, a representative delegated, entrusted or requested by the examinee.

The delivery means S230 may deliver the entire file of the highly reliable data requested for use, or deliver only part or the corresponding part of the highly reliable data. The delivery means S230 acquires the target highly reliable data and the corrected highly reliable data recorded in the highly reliable data storage database 7b or the blockchain, and delivers these to the prospective users.

By this means, for example, the first acquisition unit 302 acquires the health check information, the authentication unit 301 authenticates the health check institution to acquire the health check information, the highly reliable data generation unit 303 generates the highly reliable data of the examinee based on authentication information and the health check information, the second delivery unit 204 delivers the blocks connected by the blockchains built in the distributed network 8, and thereupon the series of operation of acquiring authentication information in the health check institution system 3 of the highly reliable data transaction system 1 according to one embodiment is finished.

<Transactions of Highly Reliable Medical Financial Data>

Also, according to one embodiment, the highly reliable data transaction system 1 may make financial institutions and/or the like handle the assignment of claims, discounts and so forth, by using billing data, which is highly reliable medical financial data, as substantial credit collateral, and, furthermore, make the health check institutions and/or the like conclude a delegation contract. Consequently, the highly reliable data transaction system 1 is able to, for example, gather a number of claims recorded in a reliable medical financial data blockchain into a fund on a certain day of each month, collect funds for providing early funding, from a plurality of cloud lenders, carry out cloud lending of highly reliable medical financial data, and provide the health check institutions with early funding. By this means, it becomes possible to collect handling fees from the creditors such as health check institutions and collect fees from the funders. Furthermore, the cloud lenders will be able to, for example, collect real interest rates.

Also, according to one embodiment, the highly reliable data transaction system 1 may collect cases that require high treatment costs due to advanced medical care and the like, record the highly reliable data and highly reliable medical financial data for a series of health check and treatment that are certified by the health check institutions in the blockchains, disclose public offering information under the examinee's consent, and provide cloud funding. Consequently, the highly reliable data transaction system 1 can promote high reliability on a wide scale, and solicit donations for treatment costs on a large scale. By this means, the highly reliable data transaction system 1 can provide records of the examinee's highly reliable data on the blockchains, to a third party who wishes to use the highly reliable data for future medical development for fees or free of charge. Furthermore, the system use fee may be collected from the examinee himself/herself and the user.

<Recording of Highly Reliable Medical Financial Data on Blockchains>

Also, in one embodiment, the highly reliable data transaction system 1 generates from the highly reliable data on the highly reliable data blockchain. Consequently, the highly reliable data transaction system 1 can ensure reliability by recording the generated highly reliable data on a blockchain. By this means, among the highly reliable data recorded on the highly reliable data blockchain, it is possible to calculate the total amount of claims such as health check fees that have the same payment date, show the same health check institution, and are handled by the same health insurance association, business, which is the employer of the examinee, or someone/some entity to do agent business for these.

Also, according to one embodiment, health check institutions such as ones that provide health checks include for example, medical care and health check institutions such as “pharmacy”, “dentistry”, “health insurance associations”, “business that employs the examinee”, “those doing agent business for these (health check fee or medical care fee debtors, or payment agent)”, and the like.

According to one embodiment, upon examination, the examinee's consent regarding the use of the examinee's highly reliable data is acquired in advance from the examinee, and the result is stored as assignment information of the highly reliable data. Consequently, it can be recorded in the blocks connected by the blockchains built in the distributed network 8. By this means, businesses (users) to use the highly reliable data can confirm, on the blockchains, that the highly reliable data of the examinee is available for transaction.

Also, according to one embodiment, the highly reliable data includes health check information, medical devices, or assignment information related to standardization. Consequently, it is possible to standardize even the results of health checks received at different health check institutions. By this means, businesses (users) to use highly reliable data can use highly reliable data of various examinees on a wide scale, and develop new services and new products. Furthermore, since it is possible to analyze highly reliable data in more detail, it is possible to propose insurance plans and services that suit the individual.

Also, according to one embodiment, the authentication of a health check institution to acquire health check information is done by the authentication information assignment system 5, which is a public institution, and the result of this authentication is recorded in the blockchains. Consequently, the credibility of the health check information can be ensured by the authentication information assignment institution. By this means, the examinee can make his/her highly reliable data available for use on a wide scale, and the user can feel safe to use the highly reliable data.

Also, according to one embodiment, the examinee sets the use condition information, the user sets the use request information, and these are recorded on the blockchains. Consequently, in the transaction management system 2, highly reliable data can be exchanged efficiently. By this means, efficient exchange of highly reliable data is enabled between the examinee and the user. Furthermore, depending on what highly reliable data the user desires to retrieve, it is possible to charge, so that the cost obtained by that charge can be efficiently returned to other health check institutions and information providers.

Also, according to one embodiment, the highly reliable data transaction system 1 may make transactions of highly reliable data, corrected highly reliable data, or corrected highly reliable data, in the transaction unit, by using or exchanging billing, electronic money, coupons, standardized tokens and the like. Standardized tokens are, for example, classified and defined under common specifications on all blockchains transacted in the highly reliable data transaction system 1. By this means, in the highly reliable data transaction system 1, this standardized token (standard token) can be interoperated even between different blockchains. in transactions in the transaction unit are using information (price, benefits) that is classified and defined by standard tokens, and, for example, in transactions of medical data, highly reliable data can be exchanged and transacted as medical tokens.

Also, according to one embodiment, the highly reliable data transaction system 1 may operate the highly reliable data agreed and authenticated by the examinee, by means of the transaction management system 2. In the transaction management system 2, the access right granting unit 203 can set access rights to the examinee's highly reliable data. Consequently, the examinee can perform transactions of highly reliable data, for example, through his/her examinee terminal 4. By this means, the examinee can, at his/her own risk, manage all the highly reliable data on applications of the examinee terminal 4.

Note that, in the highly reliable data transaction system 1, an application (not shown) may be provided on the examinee terminal 4 by an insurer such as a health insurance association. The highly reliable data transaction system 1 may, for example, add an additional reservation function to the application provided by the insurer such as a health insurance association, and appointments for medical care and health check institution may be made from the examinee terminal 4, along with the above-mentioned functions provided by the highly reliable data transaction system 1. Various data (for example, health check information, highly reliable data, highly reliable medical data, etc.) is transacted and handled in the highly reliable data transaction system 1, and may be used effectively in the evaluation unit so as to confirm and identify events that may result in refunding cases, for example, upon paying medical care and medical examination fees, because information such as information for specifying insurers such as health insurance associations, receipt information, the duration of the right to take examinations, various testing items, conventional payment funds and so forth, is incorrect. By this means, for example, the transaction unit can reduce the return rate later, and, as a result of this, increase the payment rate from insurers such as health insurance associations compared to conventional rates (for example, 80%) as much as possible.

Also, according to one embodiment, in the highly reliable data transaction system 1, for example, “0001 Examinee information” stored in the transaction management information database 2b may include an area where the examinee can register data on an optional basis. This area can be stored apart from the blockchains, for example. Although this area is not authenticated by the authentication information assignment institution, for example, the examinee may declare that the data the examinee has registered is an authenticated one. Note that the declaration may be made on the examinee's my-page via the examinee terminal 4, for example, or may be recorded in, for example, “007 Highly reliable data,” together with the data of the subject of declaration and identification information to show the date and time of declaration, such as a time stamp.

Furthermore, according to one embodiment, for example, “001 Examinee information” stored in the transaction management information database 2b may include, for example, the examinee's sex, age, address and so forth, and, in addition, include information related to the examinee's residential area, such as information about the prefecture where the examinee lives.

Also, according to one embodiment, the first delivery unit 305 or the transaction unit can, for example, receive as input highly reliable data which the examinee himself/herself provides based on his/her consent, from the examinee's my-page, via the examinee terminal 4, and therefore this highly reliable data can be used at any time, by the blocks connected by the blockchains built in the network 8. Furthermore, for example, given “007 History of use” recorded in the examinee information table of the transaction management information database 2b, the transactions status of examinee's highly reliable data can be consulted as history or transition information from the past.

Also, according to one embodiment, for example, “007 History of use,” stored in the examinee information table of the transaction management information database 2b may include contract information with any service providers (for example, life insurance companies, property damage insurance companies, etc.).

Also, according to one embodiment, for example, the exchange using smart contract technology between the examinee terminal 4 and the user terminal 6 may be performed via the examinee's my-page. The my-page identified by the examinee ID may be provided with, for example, a space for inputting smart contract-related identification information (for example, number information, etc.).

The identification information may be, for example, identification information transmitted in advance to the examinee terminal 4 from a life insurance company, a property damage insurance company and the like, which are service providers. The examinee may, for example, input this identification information from the my-page to display the details of the contract and so forth, presented by the life insurance company, the property damage insurance company and the like. The contracting contents include, for example, the contents of the highly reliable data to be delivered, compensation and the like, are provided between the examinee terminal 4 and the user terminal 6, and arrangements may be made so that, if mutual consent is obtained, an “Agree” button, displayed on the screen or elsewhere, is pressed, and the contracting contents are finalized.

Also, according to one embodiment, for example, two public keys may be handed over to a life insurance company, a property damage insurance company and so forth, which are service providers, one pertaining to highly reliable data, and the other one pertaining to separate attribute data of the examinee input by the examinee himself/herself. Consequently, the examinee himself/herself can, for example, exchange the compensation that is received for highly reliable data, and obtain in exchange the private key number to this compensation. Given this secret number, the secret key may be input on a site on the highly reliable data's user side, from the website of the life insurance company, property damage insurance company, and the like, which are service providers, and collect the compensation. In addition, in order to prevent spoofing of the examinee, a third party may be allowed to make transactions on a terminal by using the examinee's data.

Also, according to one embodiment, compensation may be provided in the form of, for example, cash back, various exchange points, the right to subscribe to a new insurance product with a good insurance premium, provision of various diversified services, and so on, and the provider of highly reliable data may appropriately make selections from the information presented on the user's web site. Also, the transaction-managing side may receive a predetermined fee or the like from, for example, a life insurance company, a property damage insurance company and the like, which are service providers that have finished transactions of highly reliable data. Furthermore, the transaction-managing side may, for example, pay a predetermined fee and/or the like, as appropriate, to the authentication information assignment institution and the health check institution.

Also, according to one embodiment, compensation may be provided in the form of, for example, cash back, various exchange points, the right to subscribe to a new insurance product with a good insurance premium, provision of various diversified services, and so on, and the provider of highly reliable data may appropriately make selections from the information presented on the user's web site. Also, the transaction-managing side may receive a predetermined fee or the like from, for example, a life insurance company, a property damage insurance company and the like, which are service providers that have finished transactions of highly reliable data. Furthermore, the transaction-managing side may, for example, pay a predetermined fee and/or the like, as appropriate, to the authentication information assignment institution and the health check institution. Also, standardized tokens may be used for transactions and payment.

Also, according to one embodiment, the examinee obtains information related to the consent for the use of examination data upon an examination (for example, decision-making information of the examinee). This consent-related information may be recorded in a block on a blockchain as various information related to the examination of the examinee.

With the consent of the examinee, for example, a signature document image and a signature image of the consent form by the examinee may be obtained together. By adding these signature document image and signature image, it may be determined that the examinee has properly agreed. The judgment as to whether the signature document image or the signature image is true may be controlled by, for example, artificial intelligence. Here, “artificial intelligence” may be based on any well-known artificial intelligence technology.

Also, according to one embodiment, highly reliable data, standardized highly reliable data, hashed highly reliable data, standardized hashed highly reliable data, and so forth may be recorded in, for example, a server built and installed by an institution such as a non-profit institution, or in blocks on a blockchain of a cloud environment. By this means, stored data's standardization, fairness, neutrality and so forth are ensured, so that it becomes possible to use the data efficiently even in situations where the conditions, settings, purpose and time of use vary.

Note that the information to be recorded in the block on the blockchain may include various data such as medical examination data, approved and standardized as reliable data by the health check institution, medical examination data, or receipt. These data are provided so as to enable a mechanism (not shown), whereby can transactions can be made, with a third party, such as another health insurance association, through for example, procedure based on the person's (examinee, insurer, etc.) direct or indirect intention, for example, via the highly reliable data transaction system 1. Furthermore, the operator of the transaction management system 2 may get a predetermined system use fee as compensation, depending on the order for data acquisition from a transacting party, the amount of transaction data, and/or the like. By this means, depending on the conditions of the data to be transacted and the amount of data, the system operator will also be paid appropriate compensation, so that it becomes possible to make transactions with a third party, efficiently, while maintaining the safety of transactions.

Also, according to one embodiment, the user can, for example, access the highly reliable data transaction system 1 via the user terminal 6, and receive various services provided by the highly reliable data storage system 7. As for these various services, for example, menu information and the like are stored in the highly reliable data storage database 7b, and the user terminal 6 having requested the use may be allowed to consult or be provided with the data or information that the user terminal 6 wants to use, through the highly reliable data storage device 7a.

Also, according to one embodiment, the highly reliable data storage system 7 may be arranged so that, for example, based on transactions of highly reliable data in the transaction management system 2, part or all of the highly reliable data is provided to the user terminal 6, in accordance with the access rights set up or licensed by the examinee, via the examinee terminal 4. The highly reliable data storage system 7 may be operated by, for example, another service company (not shown) besides a non-profit institution. In this case, for example, the non-profit institution or the service company obtains the examinee's consent, and grants access rights to the highly reliable data subject to transactions, based on the above-mentioned smart contract. The access rights may be granted, for example, based on existing authentication key technology, for transacting highly reliable data.

By this means, the examinee can benefit from managing his/her health check information for the user, by himself/herself. The value of use for the user is heightened even more, because standardized highly reliable data is provided. The health check institution (health check institution), which provides health check information, can have cash quickly. Service companies and/or the like can collect the amount of money from the user, who uses highly reliable data, according to the conditions of use of the highly reliable data, on behalf of the user. Other service companies may, for example, set a part of the amount of money collected from the user, as an appropriate data fee and handling fee, based on, for example, various information such as a fixed amount, or the type of highly reliable data to use, the amount of data, the duration of use, the method of use, the location of use, the frequency of use and so forth, to the examinee and a non-profit institution, and, by linking this with the highly reliable data to be transacted, pay this to the examinee, non-profit organization and so forth. The history of these transaction information, payment information and the like may be, for example, recorded in a block on a blockchain in association with various related information and the highly reliable data.

Also, according to one embodiment, the acquisition, authentication, judgment, or the use of highly reliable data described above may be controlled by, for example, artificial intelligence. Here “artificial intelligence” may be used in any of the above acquisition, authentication, judgement or use of highly reliable data, based on any existing artificial intelligence technology. Furthermore, based on these acquisition, authentication, judgment, or use of highly reliable data, for example, based on artificial intelligence technology, necessary data analysis is performed for various needs of users, and, depending on the results of those analysis, highly reliable data may be acquired, authenticated, judged, or used.

Also, according to one embodiment, for example, the highly reliable data transaction system 1 may appropriately calculate the compensation (benefit) in response to the delivery of various data from the health check institution or the testing institution. The health check institution or the testing institution that provides various data such as the health check results of the examinee, may calculate the medical examination fee, which used to take 2 to 3 months, based on the track data, as compensation for providing the data transacted or delivered, and acquire a claim from the health insurance association based on the calculated medical examination fee. By this means, it will be possible to monetize early, at a low cost.

Also, according to one embodiment, the receipt processing system (not shown) that cooperates with the highly reliable data transaction system 1 stores information specifying and finalizing the assignment of claims (or collateral) for a health insurance association, in association with the health insurance association's examinees, insurers, and/or the like. By this means, for example, for each claim, it is possible to create assignment contract for a financial service provider with a health check institution or a testing institution. The receipt processing system can consult the information related to the assignment contract executed, and draft the corresponding assignment contract legally, so that it becomes possible to prepare documents, such as assignment contracts, without the need for stamp taxes, which used to be a significant cost in the past.

Furthermore, the receipt processing system consults the score levels of risks (not shown) related to the insurers, who are the creditors, identifies targets where conventional costs are born, according to the insurers' score levels of as consulted, and, based on the result identified, reduces the costs of the targets where costs are born. By this means, it is possible to reduce the cost of registration.

Also, according to one embodiment, the receipt processing system may, for example, acquire the subject of registration reservation regarding transactions or delivery of various data based on, for example, transactions of highly reliable data in the transaction management system 2, and provide these to the financial service provider. By this means, to show the insurers' risk scores, financial information of the insurers and others, and other additional information are shown. Furthermore, for example, external information such as SNS information may be taken in, and the credit risk scores of the insurers may be presented as reference information by utilizing AI or the like. Furthermore, based on the claim assignment contract information, it may be possible to consult the financial statements of the health check institution or testing institution, and display each off-balance-related setting information or whether or not the conditions are met, based on the result of consulting.

<Transaction of Highly Reliable Data (Highly Reliable Medical Data)>

Also, according to one embodiment, the highly reliable data transaction system 1 may perform health checks, complete medical checkups, special health checks, dental checkups, various medical cares and so forth (health checks and the like), as highly reliable data transactions, with the consent of the examinee in advance, and generate highly reliable data. Consequently, the highly reliable data transaction system 1 can standardize the highly reliable data of the examinee, and store the standardized highly reliable data in blockchains, together with the authentication record of the health check institution and the like. By this means, the highly reliable data transaction system 1 enables transactions to be delivered to the user (third party) as highly reliable data. Furthermore, the highly reliable data transaction system 1 can, for example, exchange data already stored in the blockchains with the user, at the will of the examinee, even after the transaction with the user.

Also, according to one embodiment, the highly reliable data transaction system 1 may record authentication from the health check institution or the like, after obtaining the consent of the examinee in advance upon examination such as a health check. Consequently, the highly reliable data transaction system 1 can collect medical data such as health check result data, receipt data, billing data, and so forth, standardize the collected medical data based on a standardization model, verify it, and records it in the blockchains as highly reliable data. By this means, the highly reliable data transaction system 1 can generate highly reliable data that is standardized based on a standardization model, and deliver the generated highly reliable data to a third party who desires to use this highly reliable data. Furthermore, the highly reliable data transaction system 1 can deliver to third parties such as businesses, health insurance associations, and their agents, so that it is possible to collect an appropriate system use fee upon delivery to those third parties.

Also, according to one embodiment, the highly reliable data transaction system 1 may specify the highly reliable data that is recorded on the server or cloud recorded on the blockchain, or shown in the tracks (indices) of transactions of highly reliable data recorded on the blockchains, at the will of the examinee. Consequently, it is possible to deliver to third parties such as, for example, private insurance companies, pharmaceutical companies, health-related service providers, research institutions, health check institutions and the like, and the examinee can execute transactions to be entitled to intangible and tangible benefits in exchange, on the terminal. By this means, the examinee can record the examinee's right to receive as pre-defined points or as the balance, until they are exchanged for benefits such as cash vouchers, goods, and intangible services. Furthermore, the examinee can collect the system use fee and the advertising fee when a transaction with a third party using highly reliable data is carried out.

Also, according to one embodiment, recording in the blockchains of the highly reliable data transaction system 1 may be carried out so that all the information, including medical data such as health check results, is recorded in the blockchains, and/or part of the information including medical data such as health check results is recorded on the server or in the cloud. Consequently, all or some of the information of the medical data to be transacted is recorded on the blockchains as tracks (for example, indices). By this means, for example, highly reliable transaction data including highly reliable data makes it possible to record and acquire highly reliable data, its related information, history information and so forth, in chronological order, by using my-numbers as keys, even when the insurer changes and the insurance card changes, when the job changes, when the family name changes upon marriage, divorce, and so forth.

Also, according to one embodiment, highly reliable data includes, for example, “the name of the examinee”, “the date of birth of the examinee”, “the address of the examinee”, “the sex of the examinee”, “the my-number card number of the examinee”, “the health insurance card number of the examinee”, and “the institution where the examinee took the health check and the like (health check fee creditor)”. Also, according to one embodiment, health check institutions such as ones that provide health checks include for example, medical care and health check institutions such as “pharmacy”, “dentistry”, “health insurance associations”, “business that employs the examinee”, “those doing agent business for these (health check fee or medical care fee debtors, or payment agent)”, and the like.

Also, according to one embodiment, the highly reliable data may include “the amount billed for the health check fee”, “the due date of payment”, “the date the health check was taken”, “the health check taken”, and, besides, as related pre-standardization information, for example, “the health check result before standardization”, “the medical data before standardization”, “the receipt information before standardization”, “the standardization model”, “the presence or absence of standardization”, “the date and time of standardization” and so forth may be included. Furthermore, as information after standardization, for example, “the health check result after standardization”, “the medical data after standardization”, “the receipt information after standardization”, “the data to be read in standardization”, “the delivery information related to standardization”, “the correction information related to standardization” and “the deletion information related to standardization”. Furthermore, for operations for various kinds of information, for example, “the date and time of execution of reading/delivery/correction/deletion and so forth, and executor-specifying information, item/content information, delivery location information” and/or the like may be included.

Also, according to one embodiment, the highly reliable data transaction system 1 may generate highly reliable data from the highly reliable data stored on a blockchain. Consequently, highly reliable data can be recorded on a highly reliable blockchain, and can be used as highly reliable medical financial data for, for example, billing data for the health check fee at health check institutions where reliability is ensured. By this means, it becomes possible to provide high value-added financial services to health check institutions and elsewhere.

Also, according to one embodiment, the highly reliable data transaction system 1 may transact highly reliable medical financial data by executing, for example, assignment of claims, in which financial institutions and the like make the billing data the collateral for the claims, as well as discount transactions for discounting, cloud funding to solicit funds from a large number of people, cloud funding to solicit a large number of donations, and so forth. Consequently, the highly reliable data transaction system 1 allows, for example, a health check institution to select optimal financial services by itself, and a plurality of financial institutions to display the contents and conditions of proposed financial services on terminals, and the health check institution can analyze optimal financial services, using AI or artificial intelligence, and make proposals on the terminals, based on financial needs input (for example, the need for prioritizing an earlier deposit date, prioritizing days when the fee is set lower, prioritizing the main bank, prioritizing partners with higher funding ratios, etc.), or based on financial service history information such as past loans, end-of-fiscal-period information of the health check institution prepared in advance, and so on. By this means, the highly reliable data transaction system 1 can, for example, collect the advertising fee and system use fee from the financial institutions and/or elsewhere, depending on the result of proposals.

Also, according to one embodiment, the highly reliable data transaction system 1 may allow financial institutions and/or the like to use the billing data, which is highly reliable medical financial data, as effective credit collateral, and execute assignment or discount of claims, and, furthermore, allow health check institutions and the like to conclude a delegation contract. Consequently, the highly reliable data transaction system 1 can deliver the highly reliable medical financial data that is located on the highly reliable medical financial data blockchain, such as the names of creditors and their account information, the names of debtors and their account information, the amount of claims, the due date of payment, and so forth, to financial institutions as effective credit collateral information, for example, on a certain day of every month, and enable cooperation with related financial platforms such as the Internet. By this means, the highly reliable data transaction system 1 can process all the business operations such as, for example, having consent from the debtors, split of debt (for example, 80% or 90%, where the accuracy is calculated from the past billing return ratios of the health check institutions to determine the splitting ratio), assignment of claims, discount of claims, deposit, settlement, and so forth, within the highly reliable data transaction system 1. Furthermore, the highly reliable data transaction system 1 makes use of highly reliable medical financial data, and makes it possible to monetize to the creditors such as health check institutions reliably, quickly, and cheaply.

Also, according to one embodiment, the highly reliable data transaction system 1 may later check the billed claims against the data of the contracting contents between the health check institution and the business of its agent, the health insurance association, and so forth, returns ones that do not match, to the health check institution, finalize the final amounts of bills, and bill these to the business, the health insurance association, or the payment agent. Consequently, the highly reliable data transaction system 1 can automatically settle the amount of debt that has been monetized earlier, on the due date of payment, on a corresponding platform such as the Internet or the like, and transfer the amount of money, corresponding to the difference from the final billed amount, to the creditors such as those who have taken a health check. By this means, the highly reliable data transaction system 1 can collect handling fees from the creditors such as health check institutions, payment agent service fees from businesses or health insurance associations, and data linking fees from financial institutions. In addition, it becomes possible to handle the financial institutions' business operations for the assignment of claims, and discount of claims, and so forth, and collect payment handling fees (real interest rates, etc.).

Also, according to one embodiment, a highly reliable data transaction method with a distributed network 8 can be provided by means of an authentication means for implementing an authentication means S100, a first acquisition means for implementing a first acquisition means S110, a highly reliable data generation means for implementing a highly reliable data generation means S120, and a first recording means for implementing a first recording means S130, and a first delivery means S140 for delivering part or all of the highly reliable data to users.

Although embodiments of the present invention have been described above, these embodiment are presented as examples and are not intended to limit the scope of the present invention. Also, the present invention is limited no single unique embodiment. One embodiment can be implemented in various forms, and various omissions, replacements, and changes can be made without departing from the gist of the present invention. Such embodiments and modifications thereof are included in the scope and gist of the present invention, and are also included in the scope of the present invention described in the claims and the equivalent scope thereof.

### REFERENCE SIGNS LIST

1 highly reliable data transaction system
10 housing
100 CPU
101 ROM
102 RAM
103 storage unit
104-106 I/F
108 internal bus
109 output part
110 input part
2 transaction management system
20 acquired data
20a-20c acquired data
21 hashed data
21a-21i hashed data
201 second acquisition unit
202 receiving unit
203 access right granting unit
204 second recording unit
2a transaction management device
2b transaction management information database
3 health check institution system
301 authentication unit
302 first acquisition unit
303 highly reliable data generation unit
303a standardization process unit
304 first recording unit
305 first delivery unit
3a health check institution device
3b medical device
3c health check institution database
4 examinee terminal
5 authentication information assignment system
5a authentication information assignment device
5b authentication information assignment database
6 user terminal
7 highly reliable data storage system
7a highly reliable data storage device
7b highly reliable data storage database
8 distributed network
S100 first acquisition means
S110 authentication means
S120 highly reliable data generation means
S130 first recording means
S200 second acquisition means
S210 receiving means
S220 access right granting means
S230 delivery means
S240 second recording means
a, b, n nodes
t, t0, t-1 time

1. A highly reliable data transaction system with a distributed network, transacting a health check result of an examinee, the highly reliable data transaction system comprising:
authentication means for authenticating a health check institution, based on health check institution information related to the health check institution where the examinee takes a health check;
first acquisition means for acquiring, at least, examinee information related to the examinee taking the health check at the health check institution authenticated by the authentication means, consent information related to consent for transaction of a health check condition for the health check and a health check result of the health check, and health check result data of the examinee having taken the health check at the health check institution;
highly reliable data generation means for generating highly reliable data, subject to the transaction with a user, from the health check result data of the examinee acquired in the first acquisition means, based on authentication information indicating that the health check institution is authenticated, and the consent information;
first recording means for sorting and recording part or all of the highly reliable data, generated in the highly reliable data generation means, in a highly reliable data storage database for storing the highly reliable data, or sorting and storing the highly reliable data, generated in the highly reliable data generation means, in blocks connected by a blockchain built in the distributed network; and
first delivery means for, when transaction information related to acquisition of the part or all of the highly reliable data recorded by the first recording means is acquired, delivering the part or all of the highly reliable data to the user, depending on the transaction information.

2. The highly reliable data transaction system according to claim 1, further comprising:
second acquisition means for acquiring use condition information related to a condition of use of the highly reliable data, via an examinee terminal of the examinee;
receiving means for receiving use request information related to a use request for the highly reliable data, from a user terminal of the user requesting use of the highly reliable data;
access right granting means for granting an access right, to the user, to the part or all of the highly reliable data recorded by the first recording means, via the examinee terminal, based on the use condition information acquired in the second acquisition means and the use request information received in the receiving means; and
second delivery means for transacting the part or all of the highly reliable data, where the access right is granted by the access right granting means, based on the use condition information, the use request information and the access right, and, furthermore, delivering the part or all of the highly reliable data, to the user, depending on a result of the transaction.

3. The highly reliable data transaction system according to claim 2, wherein the highly reliable data generation means further comprises:
standardization process means for standardizing part or all of the health check result data acquired in the first acquisition means;
second recording means for recording the health check result data, standardized in the standardization process means, in the highly reliable data storage database or in the blocks connected by the blockchain built in the distributed network, as standardized health check result data; and
third delivery means for, when the transaction information related to the acquisition of the part or all of the highly reliable data recorded by the first recording means is acquired from the user, delivering the part or all of the highly reliable data to the user, depending on the transaction information.

4. The highly reliable data transaction system according to claim 1, further comprising:
a financial data recording unit for recording financial data, related to billing of a health check fee for the health check institution, in the blocks connected by the blockchain built in the distributed network related to financing of the highly reliable data;
claim information delivery means for gathering medical care claims of a same debtor, among medical care claim information of a same health check institution, based on the health check institution information, health check fee debtor information, an amount of claims, and a due date of payment, included in the financial data recorded by the financial data recording unit, and delivering these to a financial institution as one or more credit collateral information; and
transaction determining means for allowing the health check institution or an agent of the health check institution to select a financial institution where funding through the health check institution is possible, and to determine assignment of claims by the selected financial institution, discount of claims, or staff in charge of claims, depending on a result of the delivery of the credit collateral information from the claim information delivery means.

5. The highly reliable data transaction system according to claim 4, wherein the transaction determining means gathers the medical care claims of the same debtor, among the medical care claim information of the same health check institution, based on the health check institution information, the health check fee debtor information, the amounts of claims, and the due dates of payment, gathers the credit collateral information, and executes cloud lending based on one or more credit collateral information.

6. The highly reliable data transaction system according to claim 2, wherein the second acquisition means
acquires estimation information related to medical care of the examinee, together with the authentication information, from a medical care health check institution,
records content subject to support for the treatment of the examinee, in the blocks connected by the blockchain built in the distributed network, via the examinee terminal, based on the estimation information acquired in the second acquisition means and the highly reliable data of the examinee recorded by the first recording means, and
executes a transaction with a cloud founder, a cloud lender or the financial institution, based on the estimation information related to the medical care of the examinee, depending on the recorded content subject to support.

7. The highly reliable data transaction system according to claim 3, further comprising:
right acquisition means for acquiring a right to allow the examinee to receive an intangible or tangible benefit, depending on the delivery of the part or all of the highly reliable data by the first delivery means, the second delivery means, or the third delivery means; and
collecting means for collecting compensation in accordance with the right acquired in the right acquisition means, from the health check institution or the user.

8. The highly reliable data transaction system according to claim 1, further comprising third recording means for acquiring track information and history information related to a result of the delivery, based on the delivery of the part or all of the highly reliable data to the user, and recording the acquired track information and history information in the blocks connected by the blockchain built in the distributed network.

9. The highly reliable data transaction system according to claim 1, wherein the examinee information acquired in the first acquisition means comprises, at least, health check data related to the health check of the examinee, medical care data related to the medical care of the examinee, medication data related to medication of the examinee, genetic data related to the examinee, medical measurement device information related to the medical measurement device for performing the health check, fee billing data related to a fee, and insurer data related to an insurer of the examinee.

10. The highly reliable data transaction system according to claim 1,
wherein the first acquisition means acquires, from the health check institution, the examinee information of the examinee and correction information related to correction of the health check result data of the examinee,
wherein the highly reliable data generation means generates corrected highly reliable data of the highly reliable data generated, based on the correction information acquired in the first acquisition means, and
wherein the first recording means adds and records part or all of the corrected highly reliable data, generated in the highly reliable data generation means, to the highly reliable data storage database or each block connected by the blockchain built in the distributed network.

11. The highly reliable data transaction system according to claim 1, wherein the health check institution authenticated by the authentication means comprises, at least, a health check institution that provides health check data, a medical care institution that provides medical care data, a medical care institution that provides various medical data, a testing institution that provides test information, a dental institution that provides dental data, and a medicine institution that provides medicine data, or a gene institution that provides genetic data.

12. The highly reliable data transaction system according to claim 1, wherein the highly reliable data generated in the highly reliable data generation means comprises, at least, authenticator information related to an authenticator performing the authentication through the authentication means, authentication information including date/time information to show the date and time of the authentication, consent information of the examinee, health check institution information for identifying the health check institution where the examinee takes the health check, medical device information for acquiring the health check result data, assignment information related to correction of standardization, use information related to use of the highly reliable data, attribute-specific reference value comparative standard deviation data provided per examinee attribute, aging transition data of the examinee, storage location information for identifying a location where the health check result data is stored separately, my-number information for identifying the examinee, health insurance card information of the examinee, or future predicting data of the examinee.

13. The highly reliable data transaction system according to claim 12, wherein the assignment information included in the highly reliable data generated in the highly reliable data generation means comprises, at least, standard deviation correction data for the health check result data, attribute-specific reference value comparative standard deviation data, transition data, analysis data, device information or common language that serves as a standard for standardization, and standardization model data.

14. The highly reliable data transaction system according to claim 8, further comprising:
third acquisition means for acquiring the highly reliable data generated in the highly reliable data generation means;
a reference database, in which a training model, comprising a plurality of associations corresponding to weight variables between a plurality of data included in past highly reliable data acquired in advance, and a plurality of data included in reference information linked with the past highly reliable data, is stored;
evaluation means for consulting the reference database and generating an evaluation result of the highly reliable data;
notification means for extracting information to provide and a destination where the information is provided, based on the evaluation result in the evaluation means, and, furthermore, notifying the extracted destination of the evaluation result and the information to provide; and updating means for acquiring the highly reliable data and the corrected highly reliable data recorded in the blocks connected by the blockchain by the third recording means, based on the result of the notification by the notification means, updating corresponding history information, and storing as a new block.

15. The highly reliable data transaction system according to claim 14, wherein the training model is built on machine learning, using the past highly reliable data and the reference information as training data.

16. A highly reliable data transaction method with a distributed network, transacting a health check result of an examinee, the highly reliable data transaction method comprising:
an authentication step of authenticating a health check institution, based on health check institution information related to the health check institution where the examinee takes a health check;
a first acquisition step of acquiring, at least, examinee information related to the examinee taking the health check at the health check institution authenticated in the authentication step, consent information related to consent for transaction of a health check condition for the health check and a health check result of the health check, and health check result data of the examinee having taken the health check at the health check institution;
a highly reliable data generation step of generating highly reliable data, subject to the transaction with a user, from the health check result data of the examinee acquired in the first acquisition step, based on authentication information indicating that the health check institution is authenticated, and the consent information;
a first recording step of sorting and recording part or all of the highly reliable data, generated in the highly reliable data generation step, in a highly reliable data storage database for storing the highly reliable data, or sorting and storing the highly reliable data, generated in the highly reliable data generation step, in blocks connected by a blockchain built in the distributed network; and
a first delivery step of, when transaction information related to acquisition of the part or all of the highly reliable data recorded in the first recording step is acquired, delivering the part or all of the highly reliable data to the user, depending on the transaction information.

17. The highly reliable data transaction system according to claim 2, further comprising third recording means for acquiring track information and history information related to a result of the delivery, based on the delivery of the part or all of the highly reliable data to the user, and recording the acquired track information and history information in the blocks connected by the blockchain built in the distributed network.

18. The highly reliable data transaction system according to claim 3, further comprising third recording means for acquiring track information and history information related to a result of the delivery, based on the delivery of the part or all of the highly reliable data to the user, and recording the acquired track information and history information in the blocks connected by the blockchain built in the distributed network.
